SEQUENCE LISTING (4 sequences)

SEQ ID NO: 1 - DNA, 19 bp, Artificial Sequence
  Synthetic Primer
ggaactttggagcaatgga

SEQ ID NO: 2 - DNA, 22 bp, Artificial Sequence
  Synthetic Primer
tgccaatcagaaaatctaaaac

SEQ ID NO: 3 - DNA, 19 bp, Artificial Sequence
  Synthetic Primer
tgttctgtggctatcaggg

SEQ ID NO: 4 - DNA, 19 bp, Artificial Sequence
  Synthetic Primer
tgaggagttcttctgaggg

(19) United States
(12) Patent Application Publication
(10) Pub. No.: US 2014/0336184 A1
(43) Pub. Date: Nov. 13, 2014

(54) METHOD TO ENHANCE COGNITION

(71) Applicants: Baylor College of Medicine, Houston, TX (US); University of Houston, Houston, TX (US)

(72) Inventors: Mauro Costa-Mattioli, Houston, TX (US); Ping Jun Zhu, Houston, TX (US); Jeremy A. May, Pearland, TX (US)

(21) Appl. No.: 14/361,223

(22) PCT Filed: Nov. 29, 2012
(86) PCT No.: PCT/US12/67078

§ 371 Date: May 28, 2014

Related U.S. Application Data
(60) Provisional application No. 61/564,371, filed on Nov. 29, 2011.

(51) Int. Cl. C07D 513/04; C07D 495/04; C07D 498/04; C07D 487/04

(52) U.S. Cl. CPC C07D 513/04; C07D 487/04; C07D 495/04; C07D 498/04
USPC 514/224.5; 514/395; 514/387; 514/397; 514/379; 514/373; 514/250; 514/366; 514/232.8; 514/338

(57) ABSTRACT

The present invention concerns methods and compositions regarding suppression of double stranded RNA-activated protein kinase (PKR) to enhance cognition in an individual. In specific cases, an inhibitor of PKR is provided to the individual such that cognition is enhanced thereby, including by enhancing memory, for example. Kits are encompassed in certain embodiments.

### CROSS REFERENCE TO RELATED APPLICATION

This application claims priority to U.S. Provisional Application No. 61/564,371 filed on Nov. 29, 2011, which application is incorporated herein by reference in its entirety.

### TECHNICAL FIELD

The field of subject matter of the invention includes at least molecular biology, cellular biology, biochemistry, genetics, and medicine. In specific aspects, the field of subject matter of the invention includes learning and memory, long-term potentiation, neural networks, GABAergic inhibition, and/or network hypersynchrony.

### BACKGROUND OF THE INVENTION

The double stranded (ds) RNA-activated protein kinase (PKR) is widely present in vertebrates, and its activation leads to the phosphorylation of several substrates, the major known cytoplasmic target being the translation initiation factor eIF2α□ (Dever et al., 2007). Although PKR is activated in response to a variety of cellular stresses such as viral infection (Garcia et al., 2007), status epilepticus (Carnevalli et al., 2006), and in degenerating neurons in several neuropathologies, including Alzheimer's (Couturier et al., 2010; Morel et al., 2009; Peel and Bredesen, 2003), Parkinson's (Bando et al., 2005), Huntington's (Bando et al., 2005; Peel et al., 2001) and Creutzfeldt-Jakob's diseases (Paquet et al., 2009), little is known about its role in normal neuronal function.

The brain's cognitive functions are based on the coordinated interactions of large number of neurons widely distributed within the brain. A fundamental, yet unresolved, question of modern neuroscience is how this finely-coordinated activity is achieved. Although network hypersynchrony can be driven by hyperexcitable oscillatory networks (Huguenard and McCormick, 2007; McCormick and Contreras, 2001; Steriade, 2005), transient synchronizations of neuronal discharges have been proposed to be involved in memory consolidation (Beenhakker and Huguenard, 2009; Buzsaki, 2006; Girardeau et al., 2009; Paulsen and Moser, 1998). GABAergic synaptic transmission is thought to play a pivotal role in maintaining this balance: GABAergic inhibitory neurons not only suppress the activity of principal cells but also serve as a generator of oscillations in hippocampal networks (Freund, 2003; Klausberger and Somogyi, 2008; Mann and Mody, 2010; Sohal et al., 2009), which appear to be crucially involved in memory consolidation (Beenhakker and Huguenard, 2009; Buzsaki, 2006; Girardeau et al., 2009; Paulsen and Moser, 1998). Furthermore, GABAergic inhibition also contributes to the termination of these rhythmic events, thus preventing runaway excitation during epileptic network activity. However, little is known about the molecular mechanisms underlying neuronal synchrony during memory formation.

### BRIEF SUMMARY OF THE INVENTION

In embodiments of the invention, the present invention is directed to suppression of the double stranded RNA-activated protein kinase (PKR) that leads to both increased brain rhythmicity and enhanced cognition.

Embodiments of the present invention provide the first single gene model—a defect in a hitherto unstudied brain kinase, PKR—of both hypersyncronous network activity and enhanced memory. Embodiments also include a small molecule inhibitor (PKRi), which selectively inhibits PKR activity, replicates (phenocopies) the Pkr−/− phenotype, specifically enhanced the strength of synaptic connections (L-LTP) and long-term memory and increased network rhythmicity. In certain aspects of the invention, PKR regulates these processes via a selective control of GABAergic synaptic transmission, thus uncovering a novel signaling pathway that regulates brain rhythmicity, synaptic plasticity and memory storage.

In one embodiment of the invention, there is a method of enhancing cognition in an individual, comprising the step of providing to the individual a therapeutically effective amount of an inhibitor of double-stranded RNA-protein dependent kinase. In some cases, the inhibitor comprises a protein, nucleic acid, or small molecule.

In some embodiments of the invention, an individual is subject to methods and/or compositions of the present invention. In certain cases, the individual has no detectable cognitive dysfunction. In some embodiments, the individual is tested for cognitive dysfunction by routine methods in the art. Exemplary methods include the Screening Examination for Cognitive Impairment (SEFCI), the Repeatable Battery for the Assessment of Neuropsychological Status (RBANS), Rao's Brief Repeatable Battery (BRB), the complete SEP-59 Questionnaire, Selective Reminding Test, Symbol Digit Modalities Test (SDMT), Similarities Subtest, PASAT, Stroop Test, Myers-Briggs Type Indicator, Mini-Mental State Examination, and/or the PROSPER test. In other embodiments, the individual has Alzheimer's Disease, Parkinson's Disease, multiple sclerosis, Down's Syndrome, mental retardation, Autism Spectrum Disorder, Post-traumatic stress disorder, Cerebral palsy, stroke, brain damage, head injury, brain diseases, tertiary syphilis, liver disease, kidney disease, alcoholism, thyroid deficiency, muscular dystrophy, severe malnutrition, psychoses, drug abuse, meningitis, encephalitis, brain blood clot, cerebral tumor, cerebral abscess, lead poisoning, severe hypoglycemia, insulin overdosing, degenerative diseases of the nervous system, metabolic diseases, multiple infarct dementia, hypothyroidism, normal pressure hydrocephalus, vitamin B12 deficiency, lysosomal storage disease, chemotherapy, spastic quadriplegia, encephalitis, brain abscess, fetal alcohol syndrome, or is elderly. In specific embodiments, an elderly person is one that is at least 45-50 years old. In certain embodiments, an individual of any age is subjected to methods and/or compositions of the invention. In some cases, an individual is given repeated doses of the inhibitor at intervals of one or more hours, days, weeks, months, or years.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. Genetic deletion and pharmacological inhibition of PKR lead to synchronized cortical EEG activity in vivo. Traces from bilateral cortical electrodes (left hemisphere-reference=L−r; right hemisphere-reference=R−r) show abnormal spontaneous synchronous cortical activity, including solitary interictal spikes followed by brief wave discharges (a) in freely moving Pkr^{-/-} mice, but not in WT mice (b). Injection of PKR inhibitor (PKRi; 0.1 mg/kg) induces acute spiking (d) and rhythmic bursts (e) in adult WT mice. Calibration: 1 s and 200 μV. Abnormal EEG activity was absent from all WT control recordings (n=6) but present in all Pkr^{-/-} mice (n=8) and in 6 out of 7 PKRi-injected mice (recorded one hour after of PKRi-injection). By Fisher's exact test, p values were <0.001 and <0.01; respectively.

FIG. 2. Genetic deletion or pharmacological inhibition of PKR leads to synchronized hippocampal activity in slices. Population spikes were elicited by half-maximal electrical stimulation at 0.03 Hz (indicated by an arrow). Insets in a, b, c show similar averaged traces recorded before application of bicuculline. A low dose of bicuculline (2 μM) generated pronounced after-discharges in Pkr^{-/-} slices (b), or in WT slices treated with PKRi (1 μM) (c), as compared to WT slices (A). All plots represent at least five consecutive recordings. Calibrations: 2 ms and 3 mV for insets and 10 ms and 5 mV for slow traces. Under these conditions the number of evoked spikes (d) and the duration of burst (e) were increased in Pkr^{-/-} slices or WT slices treated with PKRi. Summary data illustrated in FIGS. 2a-c. Statistical significance: *p<0.05; **p<0.01.

FIG. 3. Reduced inhibitory synaptic responses in CA1 of hippocampal slices from Pkr^{-/-} mice and WT slices treated with the PKR inhibitor (PKRi). a) Sample traces (top) and summary data (bottom) show reduced frequency but no change in the amplitude of mIPSCs [recorded at holding potential −60 mV with a KCl-containing patch pipette and in the presence of the wide-spectrum glutamate antagonist kynurenic acid (2 mM) and tetrodotoxin (TTX, 1 μM] in CA1 neurons from Pkr^{-/-} mice. Traces at right (each is an average of at least 100 sIPSCs) do not differ between WT (uppermost) and Pkr^{-/-} slices (middle), as confirmed by superimposed WT and Pkr^{-/-} IPSCs (lowest). b) Similarly, in WT slices, PKRi decreased the frequency of mIPSCs (but not their amplitude). Summary data and individual events are arranged as in (a). Calibrations (a, b): 1 s and 50 pA for slow traces and 20 ms and 20 pA for fast traces. c) Evoked IPSC amplitude [recorded at holding potential of 0 mV in the presence of APV (50 μM), CNQX (10 μM) and CGP (10 μM)] as a function of stimulation intensity are shown superimposed and plotted as input/output curves. Calibration: 100 ms and 200 pA. d) IPSCs obtained by paired-pulse stimulation are superimposed (at left) after subtracting the first IPSC from paired responses recorded at 50, 100, 200 and 400 ms inter-stimulus intervals (ISIs); and corresponding plot (right): note reduced paired-pulse depression (at 50 ms) in Pkr^{-/-} slices and WT slices treated with PKRi, compared to WT slices. The ratio of inhibitory synaptic currents (IPSC₂/IPSC₁) was measured as a function of the ISI. Data are means±SEMs. Statistical significance: *p<0.05; **p<0.01.

FIG. 4. PKRi inhibits monosynaptic evoked IPSCs in slices from WT but not Pkr^{-/-} mice. Pharmacologically isolated eIPSCs recorded in the presence of 50 μM APV, 10 μM CNQX and 10 μM CGP55845 were elicited by half-maximal stimulation. PKRi bath-application reduced the amplitude of eIPSCs in WT slices (a), but not in Pkr^{-/-} slices (b). Membrane potential was held at 0 mV and whole-cell patch recordings were performed with a gluconate-containing patch pipette. Horizontal bars indicate PKRi application; inset trace (a, b) were obtained at times “a” and “b” indicated below plots. Calibrations: 50 ms and 100 pA.

FIG. 5. Excitatory synaptic transmission is unaltered in slices from Pkr^{-/-} mice or WT slices treated with PKRi. Whole-cell recordings of EPSCs were performed in slices from WT and Pkr^{-/-} mice with a gluconate-containing patch pipettes at a holding potential of −70 mV in the presence of picrotoxin (100 μM). a) Sample traces (top) and summary data (bottom) show similar frequency and amplitude of spontaneous EPSCs (sEPSCs) in slices from WT and Pkr^{-/-} mice. b) Sample traces (top) and summary data (bottom) show similar frequency and amplitude of miniature EPSCs (mEPSCs) [recorded in the presence of picrotoxin (100 μM) and TTX (1 μM)] in slices from WT and Pkr^{-/-} mice. c) PKRi (1 μM) bath application had no effect on evoked EPSCs recorded in the presence of picrotoxin (100 μM). Data are means±SEMs. Horizontal bars indicate the period of incubation with PKRi. Calibration (a, b): 1 s and 20 pA; (c): 10 ms 100 pA.

FIG. 6. Facilitated L-LTP in slices from Pkr^{-/-} mice or WT slices treated with PKRi. a) A single high frequency train (100 Hz for 1s) elicits a short-lasting early-LTP (E-LTP) in WT slices but generates a sustained late-LTP (L-LTP) in slices from Pkr^{-/-} mice (at 220 min p<0.001). b) The facilitated L-LTP in slices from Pkr^{-/-} mice was suppressed by anisomycin (at 220 min p<0.01). c) PKRi converts E-LTP into L-LTP in WT slices [at 220 min p<0.001]. A low concentration of diazepam (1 μM) prevented the induction of L-LTP in slices from Pkr^{-/-} mice [at 220 min p<0.05; d)]; but not the L-LTP-induced by four tetanic trains in WT slices [at 220 min p>0.05; (e)]. f) In WT slices, a high concentration of diazepam (50 μM) blocked L-LTP induction by four trains at 100 Hz (at 220 min p<0.05). Horizontal bars indicate the period of incubation with PKRi, anisomycin or diazepam. Data are means±SEMs. Calibrations: 5 ms and 3 mV.

FIG. 7. Enhanced spatial and fear memory in Pkr^{-/-} mice or WT mice treated with PKRi. a) Mean escape latencies as a function of training days in the Morris water maze (one trial per day). Compared to WT controls, Pkr^{-/-} mice exhibit significantly lower escape latencies by days 7 and 8 (for WT mice n=14, for Pkr^{-/-} mice n=12; *p<0.05). b) In the probe test performed on day 9, only Pkr^{-/-} mice showed preference for the target quadrant (**p<0.01). c) Contextual fear conditioning was determined by measuring freezing times prior to the conditioning (Nave, during 2 min period) and then 24 hr after training (during 3 min period). d) Auditory fear memory was assessed by measuring freezing times 24 hr post-training either before the onset of the tone (pre-CS, for 2 min) or during the tone presentation (for 3 min). Enhanced freezing 24 hr after training indicates stronger fear memory in Pkr^{-/-} mice (c, d for WT mice n=13, for Pkr^{-/-} mice n=10; *p<0.05). e) Pkr^{-/-} mice exhibited significantly faster freezing extinction in response to the context, as compared to WT littermates (for WT mice n=8, for Pkr−^{/-} mice n=9; *p<0.05). Injection of PKRi (0.1 mg/kg) immediately after training enhanced both contextual (f) and auditory fear memories (g) (for both groups n=8; *p<0.05; **p<0.01). h) The expression of the immediate-early gene Egr-1 after contextual-fear training was similar in CA1 neurons from WT and Pkr^{-/-} mice (for both groups n=6) exposed to context (CS). In contrast, in response to the training (CS+US), there was a significantly greater number of Egr-1 positive neurons in region CA1 from Pkr^{-/-} mice, compared to WT controls (**p<0.01).

FIG. 8. The lack of Ph does not alter gross brain morphology. Horizontal brain sections from WT and Pkr^{-/-} mice were stained with Nissl stain (A) and with antibodies against GAD67 (B), VGLUT1 (C), PSD95 and (D) and PKR (E). These markers show no major structural difference between WT and Pkr^{-/-} mice. Western blotting (F) demonstrates the lack of PKR in the hippocampus from Pkr^{-/-} mice.

FIG. 9. Genetic deletion of PKR leads to synchronized cortico-hippocampal EEG activity in vivo. a) Traces from bilateral cortical and hippocampal electrodes (left hemisphere-reference =L−r; right hemisphere-reference=R−r) show cortico-hippocampal aberrant patterns of neuronal hypersynchronization in freely moving Pkr^{-/-} mice. Arrows are pointing to the onset of abnormal high frequency synchronization leading to seizures in the cortex (above) and hippocampus (below).

FIG. 10. sIPSCs and electrically isolated eIPSCs are reduced in CA1 hippocampal slices from Pkr^{-/-} mice and WT slices treated with the PKR inhibitor (PKRi). a) Sample traces (top) and summary data (bottom) show reduced frequency but no change in amplitude of sIPSCs recorded at holding potential of −60 mV in the presence of kynurenic acid (1 mM) in Pkr−/− slices. Traces at right (each is an average of at least 100 events) do not differ between WT slices (uppermost) and Pkr^{-/-} slices (middle), as confirmed by superimposed WT and Pkr^{-/-} mIPSCs (lowest). b) Similarly, in WT slices, PKRi decreases the frequency but not amplitude of sIPSCs. Summary data and individual events are arranged as in a. c) Reversible elimination of sIPSCs by bicuculline in WT slices confirms their mediation by GABA_{A} receptors. d) Reduced electrically isolated eIPSCs in Pkr^{-/-} slices and WT slices treated with PKRi. Whole-cell patch recordings were performed with a gluconate-containing pipette at holding potential of 0 mV. eIPSCs were elicited in CA1 pyramidal neurons by half- maximal stimulation. Data are summarized by histograms below. *p<0.05; **p<0.01.

FIG. 11. Cumulative inhibition is reduced in slices from Pkr^{-/-} mice or WT mice treated with PKRi. A short high frequency train (5 pulses at 100 Hz) causes a rapid decay in the amplitude of population spikes in WT slices, owing to cumulative GABAergic inhibition (a), but not in slices from Pkr^{-/-} mice (b) or in WT slices treated with either the GABA_{A} receptor antagonist bicuculline (c) or PKRi (d). These data, summarized in (e), indicate that that PKR positively regulates GABAergic inhibition.

FIG. 12. PKRi specifically enhances population spikes elicited by a single stimulus in CAL PKRi did not alter the presynaptic afferent volley or the initial slope of EPSPs (a); however it enhanced the amplitude of population spikes in WT slices (b) but not in Pkr^{-/-} slices (c), demonstrating that the PKRi effect was not due to an off-target action. (d) In WT slices pre-treated with the GABA_{A} antagonist bicuculline PKRi caused no further enhancement of firing. These results indicate that PKRi increased population spikes by reducing GABAergic inhibition.

FIG. 13. Normal basal synaptic transmission in slices from Pkr^{-/-} mice. a) Input-output data show similar amplitudes of presynaptic fiber volleys over a wide range of stimulus intensities in slices from Pkr^{-/-} mice and WT littermates. b) Input-output relation of fEPSPs as a function of presynaptic fiber volley size was also similar for Pkr^{-/-} and WT slices. c) Paired-pulse facilitation of fEPSPs (reflecting enhanced synaptic transmitter release) did not differ between WT and Pkr^{-/-} slices. Plots show mean values (±SEM) of fEPSP2/fEPSP1, for various intervals of paired stimulation.

FIG. 14. L-LTP is similar in slices from WT and Pkr^{-/-} mice whereas PKRi did not further enhance L-LTP in slices from Pkr^{-/-} mice. b) In slices from WT and Pkr^{-/-} mice, L-LTP induced by four tetanic trains at 100 Hz is similar (at 220 min p>0.05). b) In Pkr^{-/-} slices, PKRi did not further potentiate LTP elicited by a single 100 Hz train (1 s) (at 220 min p<0.05). Horizontal bars indicate the period of incubation with PKRi. Data are means±SEMs. Calibrations: 5 ms and 3 mV.

FIG. 15. Pkr^{-/-} showed normal anxiety-like behavior when tested in the elevated plus maze and open field. The time (in sec) spent in the (less secure) open arm (a), the number of open arm entries (b), and the distance traveled (in cm) in the open arm (c) did not significantly differ between WT and Pkr^{-/-} mice (p>0.05). WT and Pkr^{-/-} mice show similar total distance traveled (d) and percentage of time spent in the center of the maze (e).

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. Furthermore, as used herein, the terms “including”, “containing”, and “having” are open-ended in interpretation and interchangeable with the term “comprising”.

### I. DEFINITIONS

The term “cognition” as used herein refers to the mental process of knowing, including aspects such as awareness, perception, reasoning, and judgment, including but not limited to that which comes to be known, as through perception, reasoning, or intuition; knowledge.

The term “enhancing cognition” as used herein refers to detectably improving cognition by measuring with one or more methods in the art.

The term “enhancing memory” as used herein refers to detectably improving memory by measuring with one or more methods in the art.

The term “PKR inhibitor” as used herein refers to a compound or mixture of compounds that inhibits at least partially the activity of PKR or inhibits at least partially its expression. In some embodiments, the inhibitor interferes with the kinase activity of PKR, at least partially. Kinase activity may be detected by any methods in the art, including phospho-specific antibodies against PKR or its major downstream target eIF2a, and in vitro kinase assay, for example.

When used in the context of a chemical group, “hydrogen” means —H; “hydroxy” means —OH; “oxo” means ═O; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylimino); “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); and “sulfinyl” means S—(O)— (see below for definitions of groups containing the term sulfinyl, e.g., alkylsulfinyl).

In the context of chemical formulas, the symbol “—” means a single bond, “═” means a double bond, and “≡” means triple bond. The symbol “————” represents an optional bond, which if present is either single or double. The symbol “ [symbol] ” represents a single bond or a double bond. Thus, for example, the structure

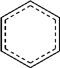

includes the structures

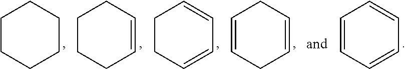

As will be understood by a person of skill in the art, no one such ring atom forms part of more than one double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation (e.g., either R or S) or the geometry is undefined (e.g., either E or Z).

Any undefined valency on an atom of a structure shown in this application implicitly represents a hydrogen atom bonded to the atom. When a group “R” is depicted as a “floating group” on a ring system, for example, in the formula:

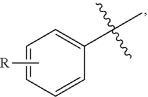

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

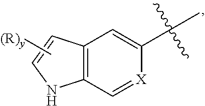

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “saturated” as used herein means the compound or group so modified has no carbon-carbon double and no carbon-carbon triple bonds, except as noted below. The term does not preclude carbon-heteroatom multiple bonds, for example a carbon oxygen double bond or a carbon nitrogen double bond. Moreover, it does not preclude a carbon-carbon double bond that may occur as part of keto-enol tautomerism or imine/enamine tautomerism.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl). When the term “aliphatic” is used without the “substituted” modifier only carbon and hydrogen atoms are present. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, P—(O)(OCH₃)₂ or —OC(O)CH₃.

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂(iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

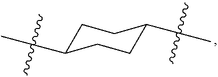 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups. An “alkane” refers to the compound H—R, wherein R is alkyl.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—,

—CH═CHCH₂—, and

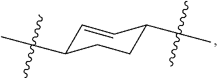 [SMILES: *C1C=CC(*)CC1]

are non-limiting examples of alkenediyl groups. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The groups, —CH═CHF, —CH═CHCl and —CH═CHBr, are non-limiting examples of substituted alkenyl groups. An “alkene” refers to the compound H—R, wherein R is alkenyl.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. The term “alkynediyl” when used without the “substituted” modifier refers to a divalent unsaturated aliphatic group, with two carbon atoms as points of attachment, a linear or branched, cyclo, cyclic or acyclic structure, at least one carbon-carbon triple bond, and no atoms other than carbon and hydrogen. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. An “alkyne” refers to the compound H—R, wherein R is alkynyl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl group (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and the monovalent group derived from biphenyl. The term “arenediyl” when used without the “substituted” modifier refers to a divalent aromatic group, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of arenediyl groups include:

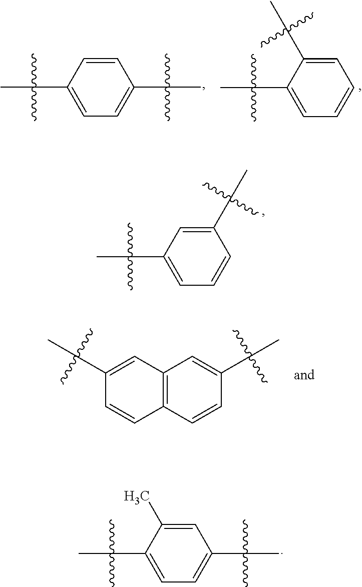

When the term “aryl” is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. An “arene” refers to the compound H—R, wherein R is aryl.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the group consists of no atoms other than carbon, hydrogen, aromatic nitrogen, aromatic oxygen and aromatic sulfur. As used herein, the term does not preclude the presence of one or more alkyl group (carbon number limitation permitting) attached to the aromatic ring or any additional aromatic ring present. Non-limiting examples of heteroaryl groups include furanyl, imidazolyl, indolyl, indazolyl (Im), methylpyridyl, oxazolyl, pyridyl, pyrrolyl, pyrimidyl, pyrazinyl, quinolyl, quinazolyl, quinoxalinyl, thienyl, and triazinyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of heteroarenediyl groups include:

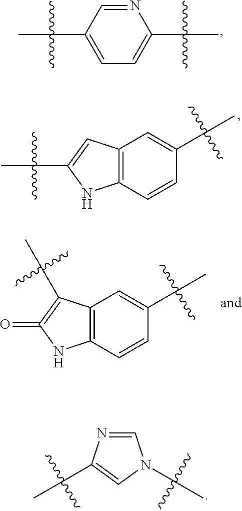

When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₁₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The terms “alkenyloxy”, “alkynyloxy”, “aryloxy”, “aralkoxy”, “heteroaryloxy”, and “acyloxy”, when used without the “substituted” modifier, refers to groups, defined as —OR, in which R is alkenyl, alkynyl, aryl, aralkyl, heteroaryl, and acyl, respectively. Similarly, the term “alkylthio” when used without the “substituted” modifier refers to the group —SR, in which R is an alkyl, as that term is defined above. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The term “alcohol” corresponds to an alkane, as defined above, wherein at least one of the hydrogen atoms has been replaced with a hydroxy group.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, and “alkylsulfonylamino” when used without the “substituted” modifier, refers to groups, defined as —NHR, in which R is alkoxy, alkenyl, alkynyl, aryl, aralkyl, heteroaryl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The groups —NHC(O)OCH₃ and —NHC(O)NHCH₃ are non-limiting examples of substituted amido groups.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” when used without the “substituted” modifier refers to the group —OP(O)(OR)(OR), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃.

The terms “alkylsulfonyl” and “alkylsulfinyl” when used without the “substituted” modifier refers to the groups —S(O)₂R and —S(O)R, respectively, in which R is an alkyl, as that term is defined above. The terms “alkenylsulfonyl”, “alkynylsulfonyl”, “arylsulfonyl”, “aralkylsulfonyl”, and “heteroarylsulfonyl”, are defined in an analogous manner. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃.

The term “heterocyclic” or “heterocycle” when used without the “substituted” modifier signifies that the compound/group so modified comprising at least one ring in which at least one ring atom is an element other than carbon. Examples of the non-carbon ring atoms include but are not limited to nitrogen, oxygen, sulfur, boron, phosphorus, arsenic, antimony, germanium, bismuth, silicon and/or tin. Examples of heterocyclic structures include but are not limited to aziridine, azirine, oxirane, epoxide, oxirene, thiirane, episulfides, thiirene, diazirine, oxaziridine, dioxirane, azetidine, azete, oxetane, oxete, thietane, thiete, diazetidine, dioxetane, dioxete, dithietane, dithiete, pyrrolidine, pyrrole, oxolane, furane, thiolane, thiophene, borolane, borole, phospholane, phosphole, arsolane, arsole, stibolane, stibole, bismolane, bismole, silolane, silole, stannolane, stannole, imidazolidine, imidazole, pyrazolidine, pyrazole, imidazoline, pyrazoline, oxazolidine, oxazole, oxazoline, isoxazolidine, isoxazole, thiazolidine, thiazole, thiazoline, isothiazolidine, isothiazole, dioxolane, thithiolane, triazole, furazan, oxadiazole, thiadiazole, dithiazole, tetrazole, piperidine, pyridine, oxane, pyran, thiane, thiopyran, salinane, saline, germinane, germine, stanninane, stannine, borinane, borinine, phosphinane, phosphinine, arsinane, arsinine, piperazine, diazine, morpholine, oxazine, thiomorpholine, thiazine, dioxane, dioxine, dithiane, dithiine, triazine, trioxane, tetrazine, azepane, azepine, oxepane, oxepine, thiepane, thiepine, homopiperazine, diazepine, thiazepine, ozocane, azocine, oxecane, or thiocane. When the term “heterocyclic” is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃.

As used herein, a “chiral auxiliary” refers to a removable chiral group that is capable of influencing the stereoselectivity of a reaction. Persons of skill in the art are familiar with such compounds, and many are commercially available.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, trifluoroacetic acid, trifluormethyl sulfonic (triflic) acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include, but are not limited to ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Effective amount,” “Therapeutically effective amount” or “pharmaceutically effective amount” means that amount which, when administered to a subject or patient for treating a disease, is sufficient to effect such treatment for the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. GENERAL EMBODIMENTS

In some embodiments of the invention, there are methods and compositions that increase cognition in an individual whether or not the individual has cognitive dysfunction. In particular, inhibitors of PKR improve cognitive function, including improve memory, such as long-term memory and/or short-term memory. In specific embodiments, the improvement is permanent. In other embodiments, the improvement is temporary but with successive administrations of the inhibitor the improvement is maintained. The inhibitor may need to be administered at certain intervals, including daily, weekly, bi-weekly, monthly, bi-monthly, or yearly, for example. The inhibitor may be administered orally, in certain embodiments.

The double stranded RNA-activated protein kinase (PKR) was originally identified as a mediator of virus infection. However, its function in the brain remains unknown. The present invention encompasses a unique mouse phenotype in which the lack of PKR leads to network hypersynchrony yet enhances long-lasting synaptic potentiation (L-LTP), memory allocation and learning and memory. In addition, administration of a selective PKR inhibitor (PKRi) to WT mice replicates the Pkr^{-/-} phenotype, namely enhanced network rhythmicity, L-LTP and memory storage. Surprisingly, these effects are caused by a selective reduction in GABAergic synaptic transmission. Hence, PKR controls the finely-tuned network activity that must be maintained while storing a given episode during learning without allowing pathological oscillations. As PKR activity is altered in several neurological disorders, PKR is a promising new target for the treatment of cognitive dysfunction.

The skilled artisan recognizes that PKR may also be referred to as EIF2AK1; MGC126524; PRKR; OTTHUMP00000201320; P1/eIF2α protein kinase; double stranded RNA activated protein kinase; eIF2α protein kinase 2; interferon-induced, double-stranded RNA-activated protein kinase; interferon-inducible RNA-dependent protein kinase; interferon-inducible eIF2α kinase; p68 kinase; protein kinase RNA-activated; protein kinase, interferon-inducible double stranded RNA dependent, or eukaryotic translation initiation factor 2-alpha kinase 2. As an exemplary illustration, PKR protein sequence is provided in GenBank® at NP_{—}002750, which is incorporated by reference herein, and the PKR mRNA sequence is provided in GenBank® at NM_{—}002759. The skilled artisan recognizes that the inhibitor of the invention may directly inhibit isoform PKR activity, eIF2α phosphorylation or indirectly promote the activity of PKR or eIF2α phosphatase.

### III. EXEMPLARY COMPOUNDS OF THE INVENTION

Compounds of the present disclosure may be made using the methods described below. These methods can be further modified and optimized using the principles and techniques of organic chemistry as applied by a person skilled in the art. Such principles and techniques are taught, for example, in March's Advanced Organic Chemistry Reactions, Mechanisms, and Structure (2007), which is incorporated by reference herein.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

In general, the compounds disclosed herein are generated through chemical synthesis by first generating an isatin, B, from a heterocycle annelated analine, A. The resulting isatin, B, is then coupled to an additinoal heterocycle through a phosphine ylide-mediated reaction to generate the structure, C. The compounds disclosed herein are generated according to the following scheme.

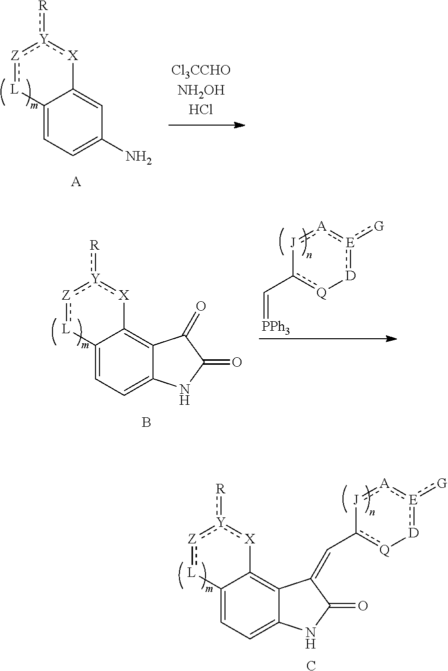

One of ordinary skill in the art would readily recognize that there are other synthetic routes to the same type of compounds disclosed herein. The above synthetic route is not limiting. The present disclosure contemplates alternate synthetic routes that yield each of the above structures all of which do not deviate from the spirit and scope of the present disclosure.

In general, X may be selected from any one of the following functional groups, such as, hydrogen (—H), hydroxy (—OH), mercapto (—SH), an oxygen atom, a sulfur atom, a nitrogen atom, a substituted nitrogen atom, a carbon atom, a substituted carbon, or carbonyl (C═O). In specific examples, X is H, OH, SH, O, S, N, NH, CH, CH₂, or C═O. In general, Z may be selected from any one of the following functional groups, such as, hydrogen (—H), hydroxy (—OH), mercapto (—SH), an oxygen atom, a sulfur atom, a nitrogen atom, a substituted nitrogen atom, a carbon atom, a substituted carbon, or carbonyl (C═O). In specific examples, Z is H, OH, SH, O, S, N, NH, CH, CH₂, or C═O. In general, L may be selected from any one of the following functional groups, such as, hydrogen (—H), hydroxy (—OH), mercapto (—SH), an oxygen atom, a sulfur atom, a nitrogen atom, a substituted nitrogen atom, a carbon atom, a substituted carbon, or carbonyl (C═O). In specific examples, L is H, OH, SH, O, S, N, NH, CH, CH₂, or C═O. In general, A may be selected from any one of the following functional groups, such as, hydrogen (—H), hydroxy (—OH), mercapto (—SH), an oxygen atom, a sulfur atom, a nitrogen atom, a substituted nitrogen atom, a carbon atom, a substituted carbon, or carbonyl (C═O). In specific examples, A is H, OH, SH, O, S, N, NH, CH, CH₂, or C═O. In general, D may be selected from any one of the following functional groups, such as, hydrogen (—H), hydroxy (—OH), mercapto (—SH), an oxygen atom, a sulfur atom, a nitrogen atom, a substituted nitrogen atom, a carbon atom, a substituted carbon, or carbonyl (C═O). In specific examples, D is H, OH, SH, O, S, N, NH, CH, CH₂, or C═O. In general, J may be selected from any one of the following functional groups, such as, hydrogen (—H), hydroxy (—OH), mercapto (—SH), an oxygen atom, a sulfur atom, a nitrogen atom, a substituted nitrogen atom, a carbon atom, a substituted carbon, or carbonyl (C═O). In specific examples, J is H, OH, SH, O, S, N, NH, CH, CH₂, or C═O. In general, R may be selected from any one of the following functional groups, such as, hydrogen (—H), hydroxy (—OH), mercapto (—SH), an oxygen atom, a nitrogen atom, or a substituted nitrogen atom. In specific examples, R is H, OH, SH, O, or NH₂. In general, G may be selected from any one of the following functional groups, such as, hydrogen (—H), hydroxy (—OH), mercapto (—SH), an oxygen atom, a nitrogen atom, or a substituted nitrogen atom. In specific examples, G is H, OH, SH, O, or NH₂.

In general, Y may be selected from any one of the following functional groups, such as, an oxygen atom, a nitrogen atom, a substituted nitrogen atom, a carbon atom, or a substituted carbon atom. In specific examples, Y is CH₂; CH, N, NH, C, or O. In general, E may be selected from any one of the following functional groups, such as, an oxygen atom, a nitrogen atom, a substituted nitrogen atom, a carbon atom, or a substituted carbon atom. In specific examples, E is CH₂; CH, N, NH, C, or O. In general, Q may be selected from any one of the following functional groups, such as, an oxygen atom, a nitrogen atom, a substituted nitrogen atom, a carbon atom, or a substituted carbon atom. In specific examples, Q is CH₂; CH, N, NH, C, or O. In general, m is 0 which forms a five-membered ring or m is 1 which forms a six-membered ring. In general, n is 0 which forms a five-membered ring or n is 1 which forms a six-membered ring.

Specific non-limiting examples of compounds generated according to the following scheme are as follows:

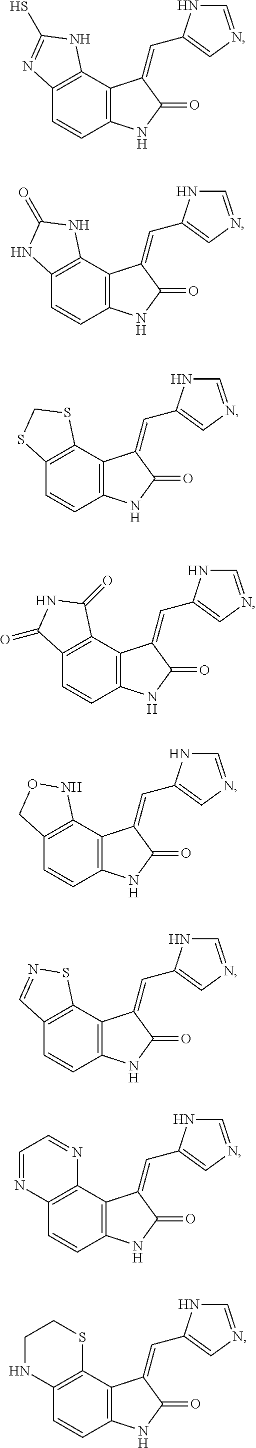

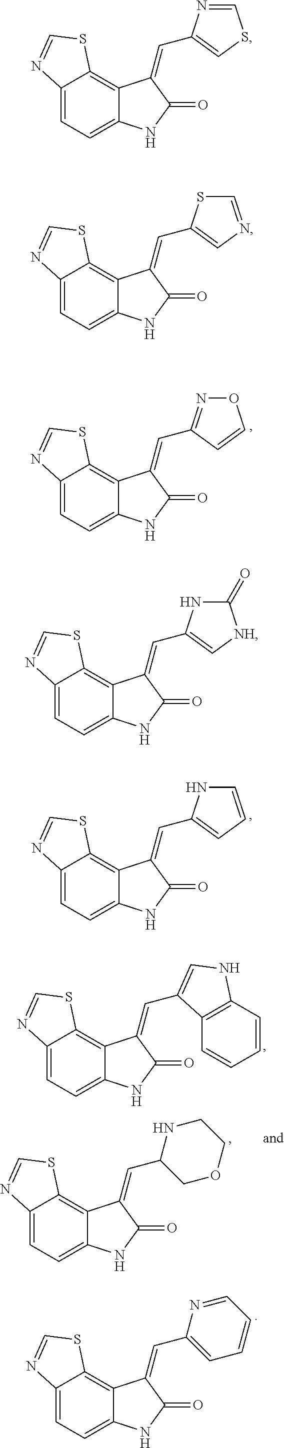

### IV. PHARMACEUTICAL PREPARATIONS

Pharmaceutical compositions of the present invention comprise an effective amount of one or more compositions of the invention dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one composition of the invention or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The PKR inhibitor may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The PKR inhibitor may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include PKR inhibitor, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the PKR inhibitor may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the composition(s) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### V. KITS OF THE INVENTION

Any of the compositions described herein may be comprised in a kit. In a non-limiting example, a PKR inhibitor is comprised in a kit in a suitable container means.

The components of the kits may be packaged either in aqueous media or in lyophilized form, for example. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the PKR inhibitor and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The composition may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. In some embodiments, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Gross Brain Morphology is not Altered in Pkr Knockout (PKR^{-/-}) Mice

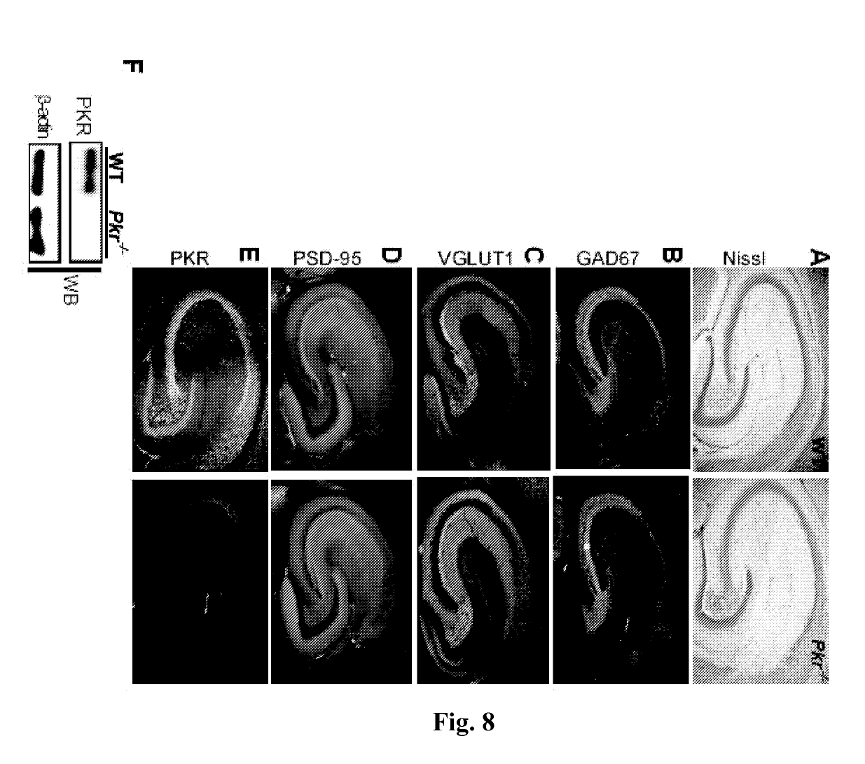

PKR knockout (Pkr^{-/-}) mice are viable, fertile and of normal size and are phenotypically indistinguishable from their wild-type (WT) littermates (Abraham et al., 2008). Nissl staining and synaptic markers for the vesicular glutamate transporter 1 (VGLUT1; a marker of pre-synaptic glutamatergic terminals), postsynaptic density protein 95 (PSD95; a marker of post-synaptic terminals) and glutamic acid decarboxylase 67 (GAD67, a marker of GABAergic terminals) show no gross abnormalities in Pkr^{-/-} mouse brain (FIG. 8). PKR is normally expressed in pyramidal cells and interneurons throughout the hippocampus (FIG. 8e). As expected PKR protein is undetectable in Pkr^{-/-} brain, as determined by immunohistochemistry and Western blotting (FIGS. 8e, f). Since PKR is relatively less abundant in the mammalian brain (compared to the other eIF2α kinases (Costa-Mattioli et al., 2009)), it is not surprising that eIF2 α □ phosphorylation is not altered in the hippocampus from Pkr^{-/-} mice (FIG. 8f).

### Example 2

### Genetic Deletion or Pharmacological Inhibition of Pkr Leads to Synchronous Network Discharges In Vivo and In Vitro

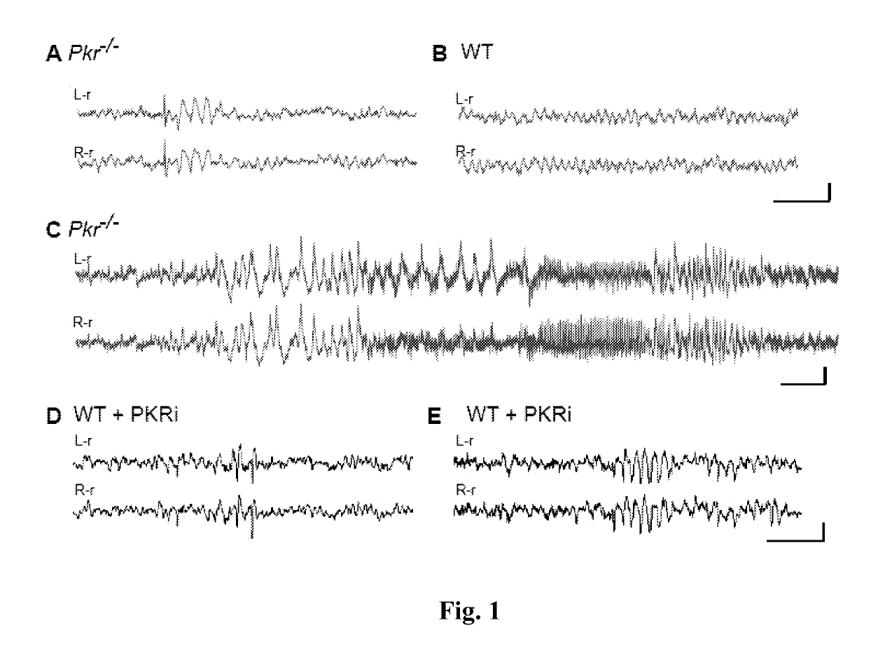

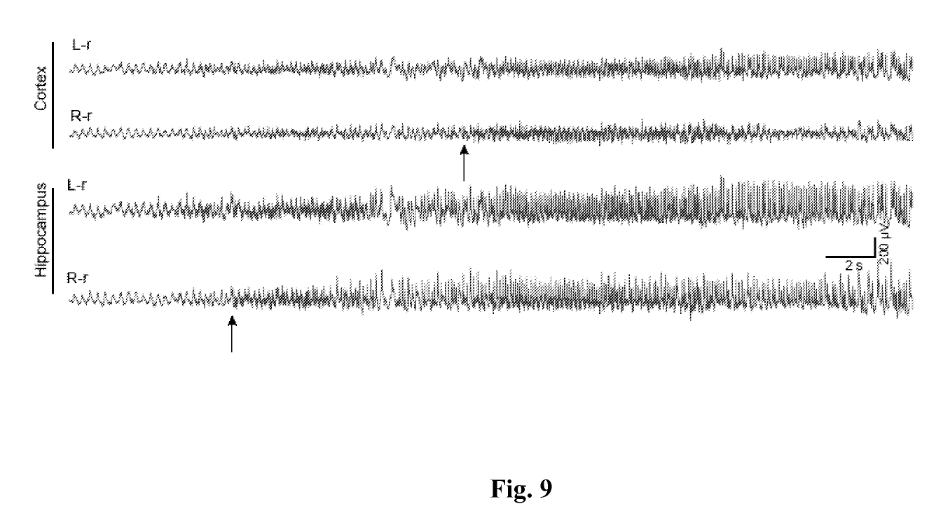

Unexpectedly, spontaneous hippocampal and cortical brain rhythms monitored in freely moving Pkr^{-/-} mice by video electroencephalography (EEG) revealed intermittent abnormal spike discharges (FIG. 1a and FIG. 9a) that were not accompanied by overt behavioral manifestations. Neither abnormality appeared in recordings from WT mice (FIG. 1b and FIG. 9b). An atypical feature of the interictal events was that instead of a solitary spike, the events consisted of a spike followed by a repetitive wave after-discharge, suggesting a deficiency in post spike inhibition. As this excitability imbalance in Pkr^{-/-} mice might arise during development, the inventors suppressed PKR activity in adult WT mice by injecting systemically a selective PKR inhibitor (PKRi) (Jammi et al., 2003). Acute PKRi administration induced both interictal spikes (FIG. 1d) and abnormal EEG rhythmic bursting activity (FIG. 1e), similar to those occurring spontaneously in Pkr^{-/-} mice (compare FIG. 1e to FIG. 1a). These observations reveal a pivotal new role for this kinase as a regulator of neuronal network rhythmicity.

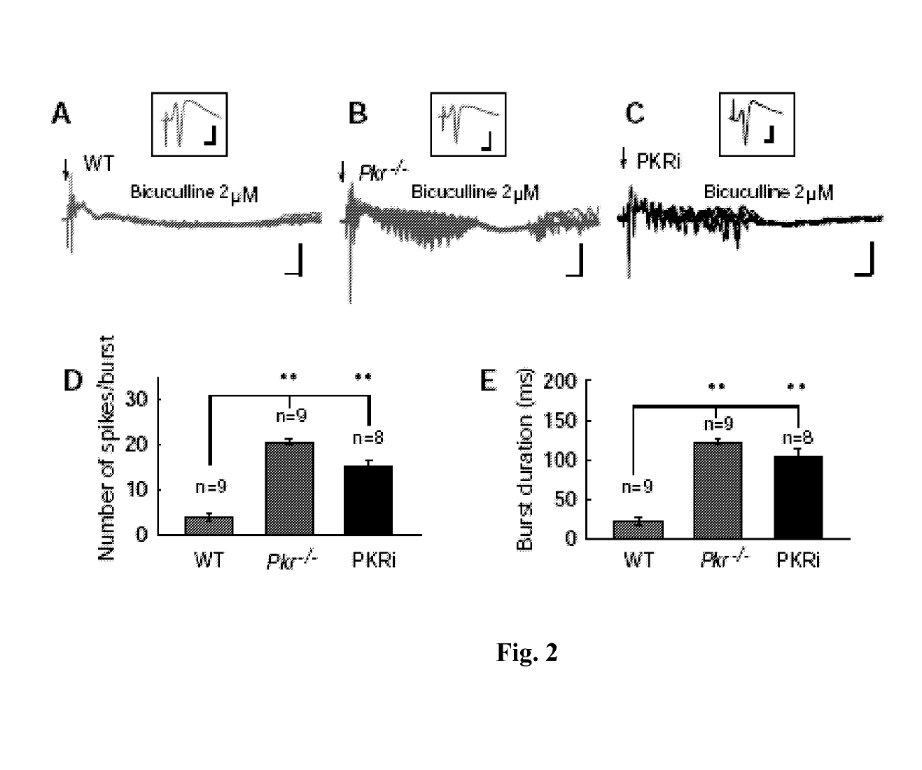

To determine whether the synchronous network activity in Pkr^{-/-} slices or WT mice treated with PKRi can be recapitulated in vitro, the inventors recorded field responses (in CA1) in hippocampal slices from WT, Pkr^{-/-} mice or in WT slices treated with PKRi. A single electrical stimulus to stratum radiatum evoked a similar field EPSP and population spike in slices from WT and Pkr^{-/-} mice (FIG. 2a, b; insets). However, in the presence of a very low concentration of bicuculline (2 μM), the same stimulus evoked a prominent after-discharge only in slices from Pkr^{-/-} mice (compare FIG. 2a to FIG. 2b, see also FIGS. 2d, e), revealing a latent hyperexcitability of hippocampal networks in Pkr^{-/-} slices. Furthermore, a similar effect was obtained when PKRi was applied to slices from WT mice (FIG. 2c; see also FIG. 2d, 2e), demonstrating that a comparable latent hyperexcitability was also induced when PKR was inhibited pharmacologically.

### Example 3

### Genetic Deletion or Pharmacological Inhibition of PKR Leads to Reduced Inhibitory Synaptic Transmission

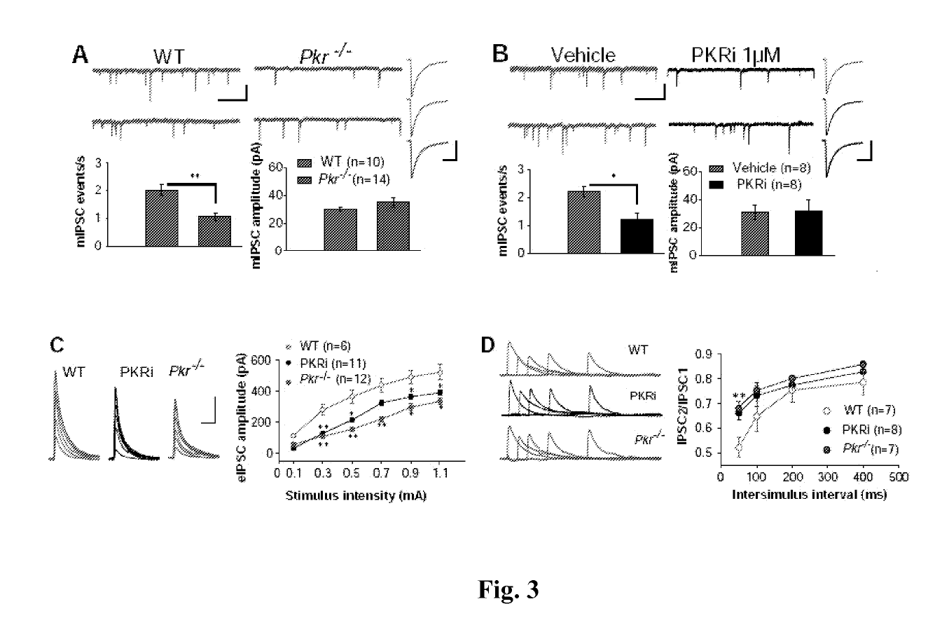

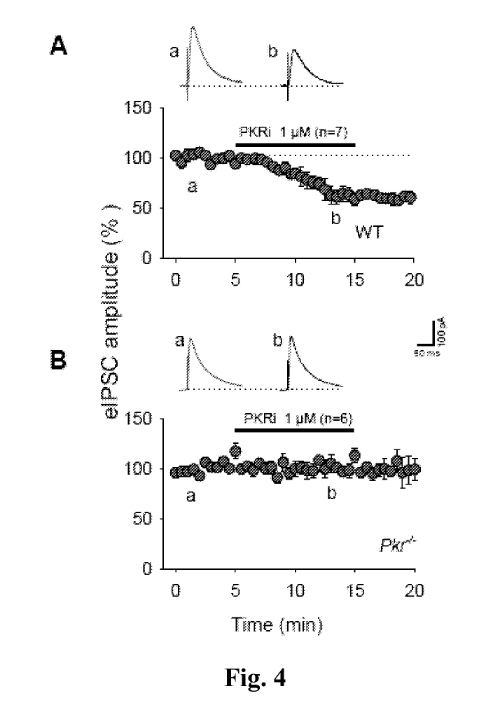

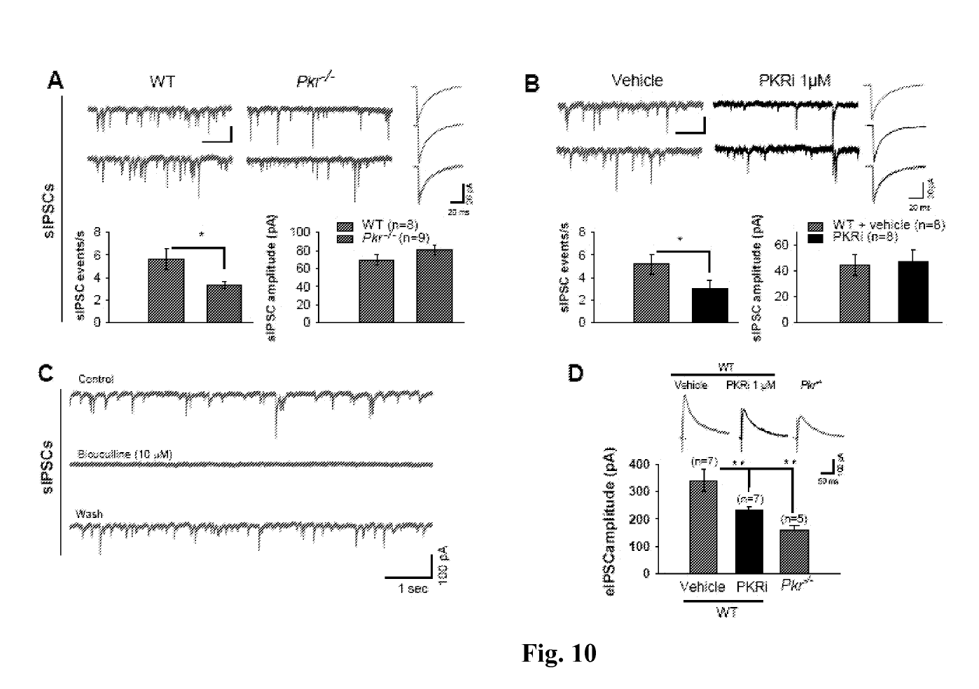

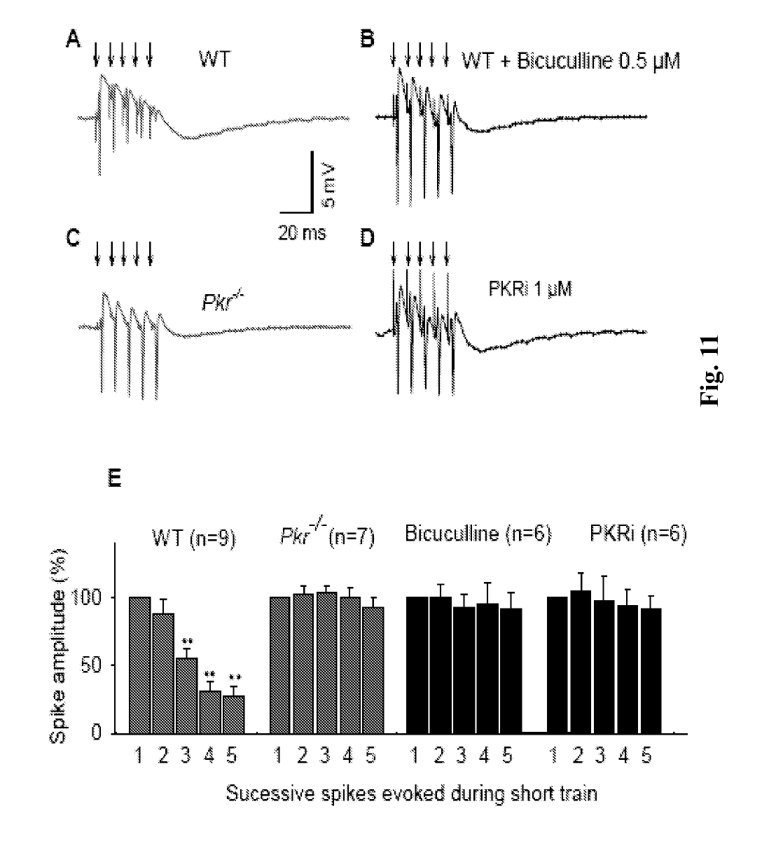

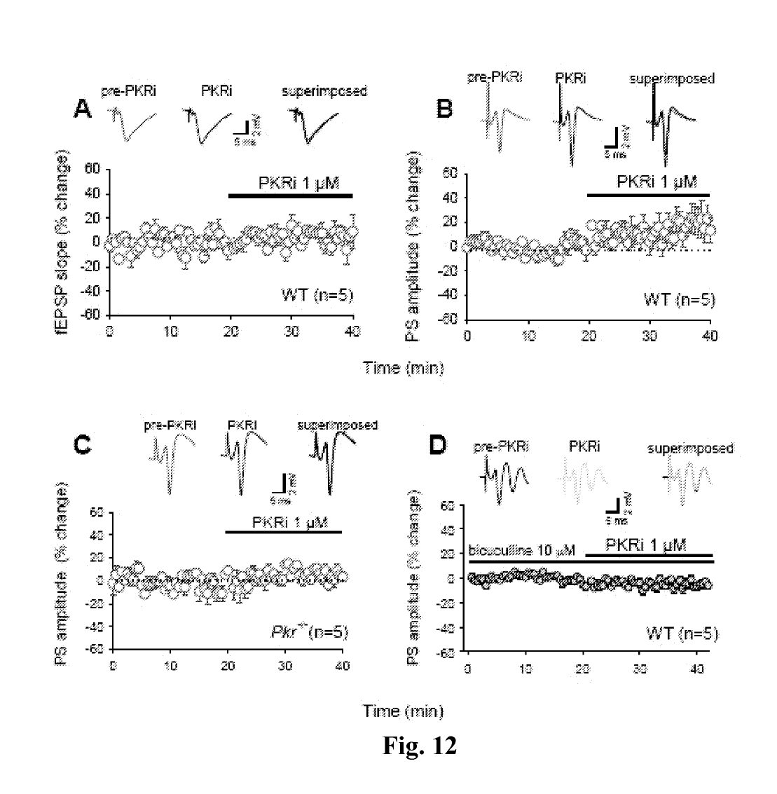

Since impaired inhibition is a common feature of genetic models of epilepsy (Noebels, 2003), the inventors considered whether it might account for the hypersynchronous activity observed in Pkr^{-/-} mice. To further characterize this, inhibitory synaptic transmission was studied in a series of experiments on hippocampal slices from WT, Pkr^{-/-} mice and WT mice treated with PKRi. First, in whole-cell patch clamp recordings from CA neurons the frequency (but not the amplitude) of both spontaneous and miniature inhibitory postsynaptic currents (sIPSCs and mIPSCs) was significantly reduced in Pkr^{-/-} slices (FIG. 3a and FIG. 10a) or in WT slices treated with PKRi (FIG. 3b and FIG. 10b). The absence of change in mIPSC amplitude is a strong indication that PKR does not affect the sensitivity of pyramidal cells to synaptically released GABA; so the reduction in ongoing GABAergic activity—indicated by the decreased frequency of mIPSCs—is likely to be caused by depression of GABA release (as supported by the results in FIG. 3d). Second, in CA1 neurons from Pkr^{-/-} mice and in WT slices treated with PKRi, the amplitude of evoked IPSCs—isolated either electrically [i.e., holding the membrane potential at 0 mV (FIG. 10c)] and/or pharmacologically (by blocking glutamate-mediated EPSCs)—was reduced over a wide range of stimulation intensities (FIG. 3c). Moreover, in contrast to its effect in WT slices, PKRi did not alter the amplitude of evoked IPSCs in slices from Pkr^{-/-} mice (compare FIG. 4a to FIG. 4b), confirming that the effect of PKRi was not due to an off-target action. Third, paired-pulse depression, a sensitive index of changes in evoked GABA release (Thomson, 2000), was significantly decreased in slices lacking PKR as well as in those treated with PKRi (FIG. 3d), indicating that PKR regulates GABA release probability. Strikingly, PKR appears to regulate inhibitory transmission pre-synaptically rather than post-synaptically as there was no difference in the rise time or decay time constant of sIPSCs and mIPSCs in slices from WT, Pkr^{-/-} mice or WT mice treated with PKRi (FIGS. 3a, b, FIG. 10 and Table 1), which is consistent with no change in postsynaptic receptors-related mechanisms. Fourth, in slices from WT mice, the amplitude of CA1 population spikes rapidly decreased during a short train of high frequency stimulation (FIG. 11a, Ile). The GABA_{A} antagonist bicuculline largely suppressed this sharp decline, which was evidently due to cumulative synaptic inhibition (FIG. 11b, 11e). In contrast, in slices from either Pkr^{-/-} or WT mice treated with PKRi there was minimal or no high-frequency stimulation-induced decrease in spike amplitude (FIG. 11c-e). These data provide further evidence that GABAergic inhibition is less efficient when PKR's function is blocked. Fifth, although PKRi had no effect on the afferent volley and the initial slope of field EPSPs in WT slices (FIG. 12a), it enhanced the amplitude of population spikes (FIG. 12b), as would be expected if the excitability of pyramidal neurons was increased as a result of reduced inhibition. In addition, PKRi had no effect on population spikes in slices from Pkr^{-/-} mice, where PKRi's target (PKR) was absent (FIG. 12c) or when GABAergic synaptic transmission was already blocked (FIG. 12d). Taken together these data provide strong genetic and pharmacological evidence that PKR selectively enhances GABAergic synaptic transmission.

TABLE 1
Properties of sIPSCs and mIPSCs in CA1 neurons from WT,
Pkr−/− and WT slices treated with PKRi.

| | Rise Time 10%-90% | |
|---|---|---|
| | (ms) | τW (ms) |
| sIPSCs | | |
| WT | 1.70 ± 0.09 | 23.29 ± 1.013 |
| Pkr^{-/-} | 1.66 ± 0.08 | 22.35 ± 0.62 |
| PKRi | 1.68 ± 0.18 | 22.89 ± 0.78 |
| mIPSCs | | |
| WT | 1.41 ± 0.1 | 20.60 ± 1.01 |
| Pkr^{-/-} | 1.38 ± 0.06 | 21.15 ± 0.81 |
| PKRi | 1.36 ± 0.07 | 21.28 ± 1.24 |

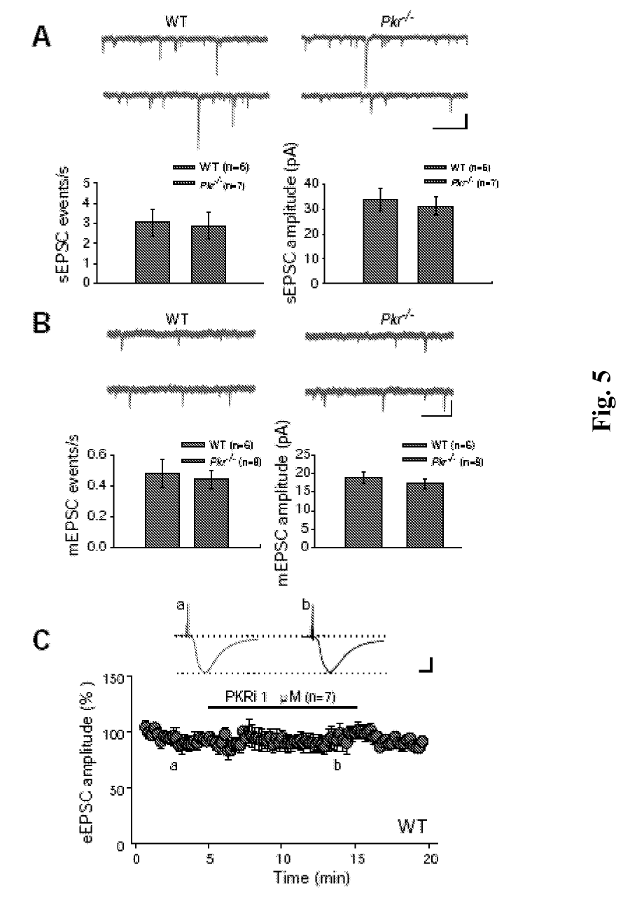

Strikingly, PKR specifically modulates inhibitory synaptic transmission since the amplitude or frequency of either spontaneous excitatory post-synaptic currents (sEPSCs), miniature EPSCs (mEPSCs) or evoked EPSCs (eEPSCs) was not significantly changed in slices from Pkr^{-/-} mice or WT slices treated with PKRi (FIG. 5).

### Example 4

### Genetic Deletion or Pharmacological Inhibition of PKR Facilitates L-LTP

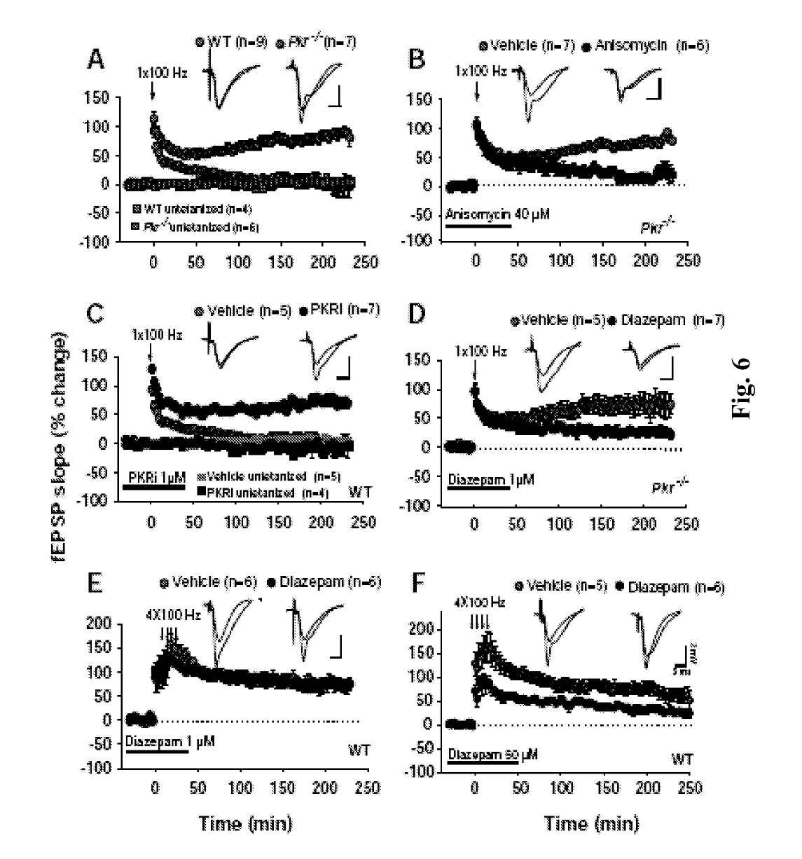

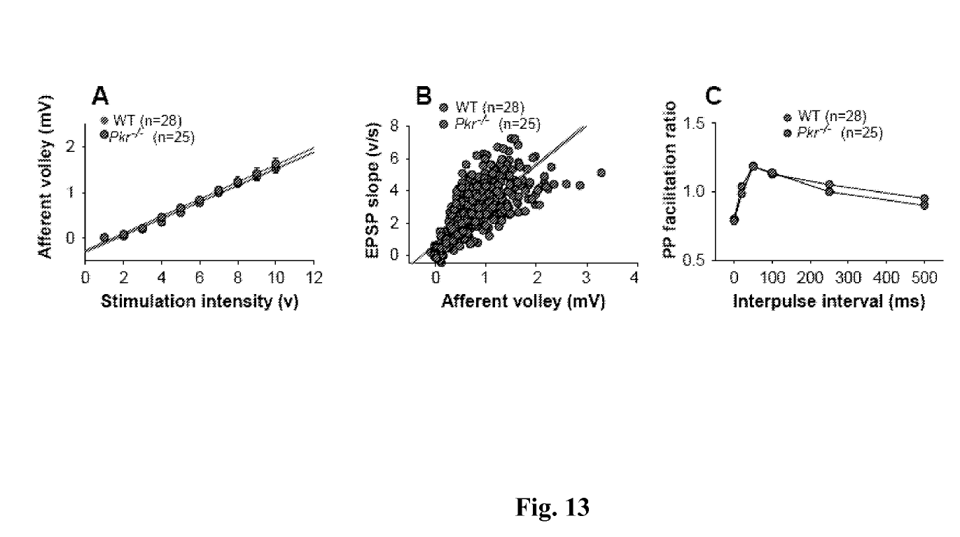

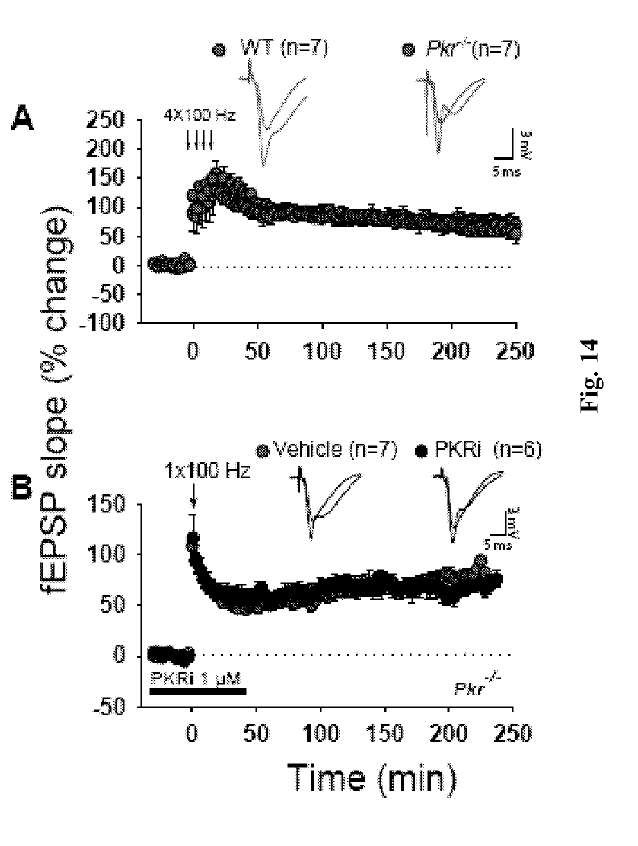

Because the induction of long-term potentiation (LTP) is facilitated by a decrease in GABA tone (Abraham et al., 1986; Davies et al., 1991; Wigstrom and Gustafsson, 1983), the inventors addressed whether reduced synaptic inhibition in slices from Pkr^{-/-} mice or from WT slices treated with PKRi could enhance the induction of LTP. Early LTP (E-LTP), which is typically induced by a single train of high-frequency (tetanic) stimulation, lasts only 1-2 hr and depends on modification of pre-existing proteins, while late-LTP (L-LTP), generally induced by several (typically four) tetanic trains separated by 5-10 min, persists for many hours and requires new protein synthesis (Kandel, 2001). In WT slices, a single high frequency stimulus train (100 Hz for is) elicited only a short-lasting protein synthesis-independent potentiation E-LTP (FIG. 6a). By contrast, in slices from Pkr^{-/-} mice the same stimulation generated a long-lasting late-LTP (L-LTP) (FIG. 6a), which was blocked by the protein synthesis inhibitor anisomycin (FIG. 6b). However, four tetanic trains (at 100 Hz) elicited a similar L-LTP in slices from WT and Pkr^{-/-} mice (FIG. 14a). The facilitation of L-LTP in slices from Pkr^{-/-} mice is unlikely to be due to changes in basal synaptic transmission since the input-output relationship of field EPSPs (as a function of the stimulus intensity), the magnitude of paired-pulse facilitation (PPF), and the size of the afferent fiber volley did not significantly differ between slices from Pkr^{-/-} and WT mice (FIG. 13). In agreement with the findings in Pkr^{-/-} slices, incubation with PKRi converted a transient E-LTP into a sustained L-LTP (FIG. 6c) in WT slices but did not induce any further potentiation in slices from Pkr^{-/-} mice, confirming the specificity of the PKR inhibitor (FIG. 14b). These data demonstrate that genetic deletion or pharmacological inhibition of PKR lowers the threshold for the induction of L-LTP.

If L-LTP is facilitated in slices from Pkr^{-/-} mice owing to reduced inhibition, reinforcing GABAergic tone in these slices should convert the effect of a single train from long-lasting to short-lasting. Indeed, incubation with a low concentration of diazepam (11 μM), which potentiates GABA_{A} action (Haefely, 1990), markedly reduced the L-LTP in slices from Pkr^{-/-} mice (FIG. 6d) but had no effect on L-LTP induced by four tetanic trains in WT slices (FIG. 6e). A far higher concentration of diazepam (50 μM) was required to prevent L-LTP in slices from WT mice (FIG. 6f). These data confirm our hypothesis that the facilitated L-LTP in slices from Pkr^{-/-} mice is a consequence of decreased GABAergic tone.

### Example 5

### Genetic Deletion or Pharmacological Inhibition of PKR Enhances Learning and Memory

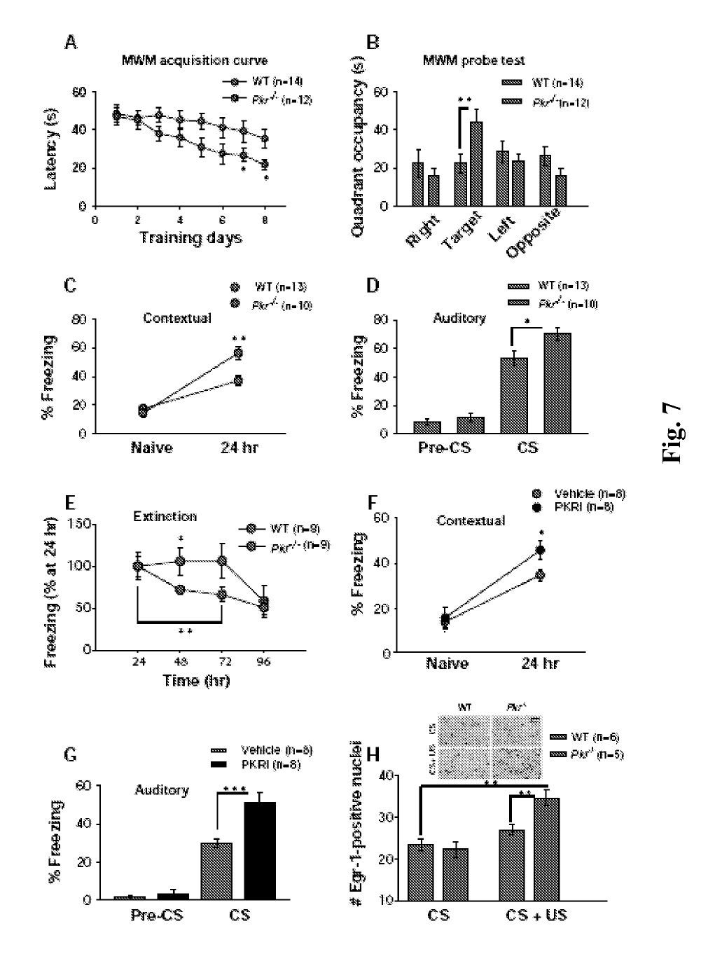

GABAergic function plays a crucial role in memory consolidation (Izquierdo and Medina, 1991; McGaugh and Roozendaal, 2009). The inventors considered whether learning and memory could be enhanced in Pkr^{-/-} mice, in which hippocampal GABA-mediated inhibition is reduced. First, mice were tested for hippocampus-dependent spatial memory in the Morris water maze, where animals use visual cues to find a hidden platform in a circular pool (Morris et al., 1982). As weak tetanic stimulation (one train at 100 Hz) revealed a long-lasting LTP in slices from Pkr^{-/-} mice, the inventors trained mice using a weak protocol (only one training session per day) for 8 days. Pkr−/− mice found the platform significantly faster than did WT control littermates (FIG. 7a); and in the probe test, performed on day 9, when the platform was removed, only Pkr^{-/-} mice remembered the platform location (targeted quadrant) (FIG. 7b). Thus, genetic deletion of PKR strengthens long-term spatial memory.

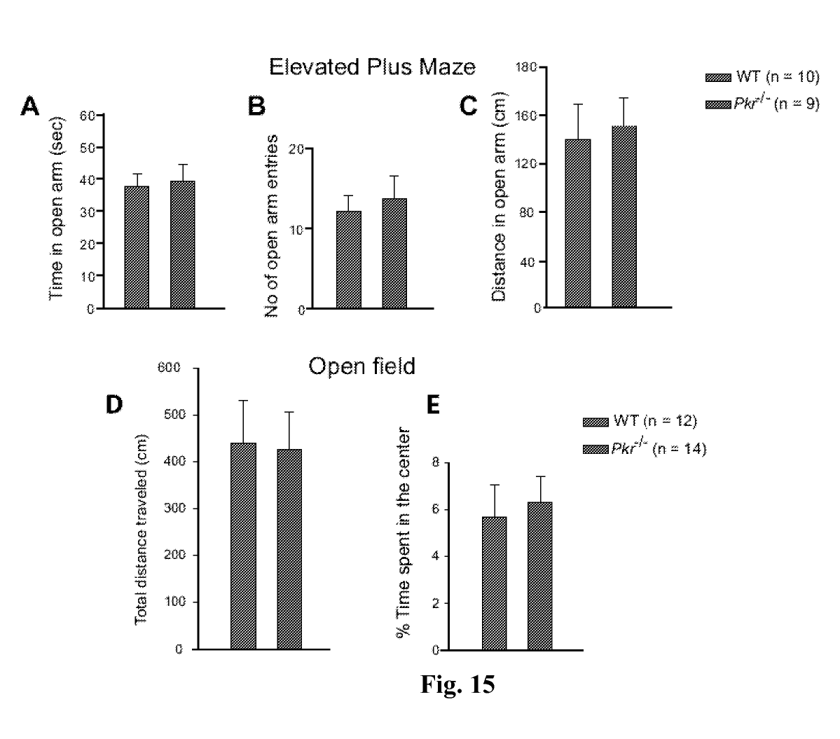

Mice were also studied in two forms of Pavlovian fear conditioning. Contextual fear conditioning was induced by pairing a context (conditioned stimulus; CS) with a foot shock (the unconditioned stimulus; US), whereas in auditory fear conditioning the US was paired with a tone presentation (CS). Contextual fear conditioning involves both the hippocampus and amygdala, whereas auditory fear conditioning requires only the amygdala (LeDoux, 2000). When mice were subsequently exposed to the CS, fear responses (“freezing”) were taken as an index of the strength of the CS-US association. Although naïve WT and Pkr^{-/-} mice showed a similar amount of freezing prior to a weak training protocol (a single pairing of a tone with a 0.35 mA foot shock), Pkr^{-/-} mice exhibited more freezing than did WT control littermates when tested 24 hr later (FIG. 7c). Similarly, Pkr^{-/-} mice showed enhanced long-lasting auditory fear memory (FIG. 7d). A non-specific response to fear in Pkr^{-/-} mice is unlikely since baseline freezing (FIG. 7c, 7d) and anxiety-reflecting behavior in both the elevated plus maze and open field (FIG. 15) was normal for Pkr^{-/-} mice. Hence, the lack of PKR improves both auditory and contextual long-lasting fear memories.

Enhanced cognition is also associated with rapid memory extinction (Lee and Silva, 2009) when animals are re-exposed (over several trials) to the test context no longer paired with a foot shock. Accordingly, Pkr^{-/-} mice showed faster extinction than did WT controls (FIG. 7e).

If PKR is involved in cognitive processing, acute pharmacological inhibition of PKR should also potentiate long-term fear memories. To test this prediction, WT mice were injected with either vehicle or PKRi immediately after Pavlovian fear conditioning. Indeed, both contextual and auditory fear memories were enhanced in PKRi-treated mice when measured 24 hr after training (FIGS. 7f, 7g).

Since PKR deficiency enhanced long-term memory storage, memory “allocation”—the process by which neurons or synapses are specifically activated (or incorporated) in a neural circuit during learning (Silva et al., 2009)—might also be enhanced in Pkr^{-/-} mice. To identify neurons selectively activated and hence participating in the encoding of fear learning, the inventors analyzed the expression of the immediate-early gene Egr-1 (also called Zif/268). Egr-1 has been extensively used for this purpose (Frankland et al., 2004; Hall et al., 2000) and its deletion blocks L-LTP and memory consolidation (Jones et al., 2001). WT and Pkr^{-/-} mice were subjected to a weak fear-conditioning protocol (a single pairing of a tone with a 0.35 mA foot shock) and the expression of Egr-1 in the CA1 region was quantified by immunohistochemistry, as previously described (Frankland et al., 2004). Egr-1 expression was not significantly different when animals of both genotypes were exposed to the context alone. In contrast, a weak training paradigm increased Egr-1 levels (and presumably memory allocation) only in CA1 neurons from Pkr^{-/-} mice (FIG. 7h) and triggered a more robust long-lasting memory in Pkr^{-/-} mice, compared to WT littermates (FIG. 7c). Thus, the lack of PKR favors the recruitment of hippocampal neurons into the encoding process.

### Example 6

### Significance of Certain Embodiments of the Invention

The present invention provides novel genetic, physiological, pharmacological, behavioral and molecular evidence that PKR negatively regulates brain rhythmicity, synaptic plasticity and memory storage by potentiating GABAergic synaptic transmission. GABAergic inhibition not only controls the efficacy and plasticity of excitatory synaptic inputs to pyramidal cells but it synchronizes firing of large assemblies of principal cells at certain preferred frequencies (Mann and Paulsen et al., 2007). Slow theta and faster gamma oscillations and ripples appear to be crucially involved in mnemonic processes (Buzsaki, 2006; Maurer and McNaughton, 2007). Several lines of evidence support the idea that GABAergic control of synaptic plasticity is a key mechanism of memory storage (Paulsen and Moser, 1998; Mann and Paulsen, 2007). First, reduced GABAergic-mediated inhibition facilitates the induction of LTP (Abraham, 1986; Davies et al., 1991; Wigstrom and Gustafsson, 1983). Second, long-term disinhibition of a subset of CA1 pyramidal neurons correlates with the acquisition of spatial memory (Gusev and Alkon, 2001). Third, modest pharmacological reduction of GABAergic transmission enhances memory consolidation (Izquierdo and Medina, 1991; McGaugh and Roozendaal, 2009). Finally, GABAergic neurons of the medial septum drive theta rhythmicity in the hippocampal network (Hangya et al., 2009), which critically contributes to hippocampus-dependent memory processes (Buzsaki, 2006).

How could the lack of PKR promote brain rhythmicity and at the same time enhance LTP and cognitive performance? In some embodiments both are a consequence of increased excitability. When PKR activity is inhibited (genetically or pharmacologically), desinhibition enhances synaptic plasticity and facilitates long-term memory storage, probably through synchronized activity in neural networks (Beenhakker and Huguenard, 2009; Buzsaki, 2006; Girardeau et al., 2009; Sohal et al., 2009; Maurer and McNaughton, 2007; Shirvalkar et al., 2010).

A byproduct of this chronic, albeit moderate, weakening of inhibition is an increased risk of electrographic seizures, in specific embodiments. Yet disinhibition in Pkr^{-/-} brain remains below the threshold for pathological seizures that could impair plasticity and memory processes. Thus PKR controls the finely-tuned network rhythmicity that must be optimized to store a given episode during learning without crossing the line into aberrant or runaway excitation.

Increased neuronal excitability appears to be a key feature of memory allocation in neurons. According to recent reports, a selective enhancement in neuronal excitability by CREB reflects the allocation and storage of fear memories in the amygdale (Han et al., 2007; Han et al., 2009; Zhou et al., 2009). During weak training, which specifically enhances memory in Pkr^{-/-} mice or WT mice treated acutely with PKRi (FIG. 7), only Pkr^{-/-} mice showed selective neuronal activation and recruitment into the memory trace (FIG. 7h). Thus, when PKR is inhibited, neuronal excitability is enhanced and neurons firing synchronously encode a given episode during a learning paradigm.

In conclusion, the data reveal that the lack of Pkr results in a novel experimental mouse of epilepsy where network hypersynchrony and enhanced long-lasting synaptic plasticity and cognition coexist. Finally, PKR's role in optimizing higher brain functions indicate that agents that inhibit PKR are therapeutically useful in the treatment of human conditions associated with memory loss, such as Alzheimer's disease, where PKR activity is abnormally elevated (Couturier et al., 2010; Peel and Bredesen, 2003; Chang et al., 2002) and GABAergic transmission is disturbed (Palop et al., 2007).

### Example 7

### Exemplary Methods and Materials

Pkr^{-/-} mice

Pkr knockout (Pkr^{-/-}) mice (Abraham et al., 1999) were back-crossed for at least eight generations to 129SvEv mice. Mice were weaned at the third postnatal week and genotyped by PCR. Briefly, the mutant and corresponding WT alleles are detected by a four-primer PCR assay in which Oligo-1 (5′-GGAACITrGGAGCAATGGA-3′) and Oligo-2 (5′-TGCCAATCAGAAAATCTAAAAC-3′) give a WT band of 225 base-pair fragment and Oligo-3 (5′-TGTTCTGTGGCTATCAGGG-3′) and Oligo-4 (5′-TGAGGAGTTICTTCTGAGGG-3′) give a 432 base-pair fragment from the deleted allele. eIF2α^{+/S51A} mice were previously described (Costa-Mattioli et al., 2007; Scheuner et al., 2001). All experiments were performed on 8-16 weeks old males. The mice were kept on a 12 h light/dark cycle, and the behavioral experiments were always conducted during the light phase of the cycle. The mice had access to food and water ad libitum, except during tests. Animal care and experimental procedures were performed with approval from the animal care committees of Baylor College of Medicine. Chronic electroencephalographic (EEG) recordings [0133]EEG recordings were performed as described (Price et al., 2009). WT and Pkr^{-/-} mice were anesthetized with Avertin (1.25% tribromoethanol/amyl alcohol solution, i.p.) at a dose of 0.02 ml/g. Teflon-coated silver wire electrodes (120 μm diameter) soldered to a microminiature connector were implanted bilaterally into the subdural space over frontal, central, parietal, and occipital cortices. Digitalized EEG data were obtained daily for up to two weeks during prolonged and random 2 hr sample recordings (Stellate Systems, Harmonie software version 5.0b).

PKRi (Calbiochem, San Diego), a potent ATP-binding-site-directed inhibitor of PKR which blocks PKR autophosphorylation (Jammi et al., 2003; Shimazawa and Hara, 2006), was prepared as a 20 mM stock solution in DMSO (dimethyl sufloxide). PKRi was freshly dissolved in saline and then injected intraperitoneally (i.p.) at a dose of 0.1 mg/kg and the EEG was recorded 1 hr after injection. A digital video camera simultaneously monitored behavior during the EEG recordings. All recordings were done at least 24 hr after surgery on mice freely moving in the test cage.

### Electrophysiology

Field recording: horizontal hippocampal slices (350 mm) were cut from brains of WT or age-matched Pkr^{-/-} littermates in 4° C. artificial cerebrospinal fluid (ACSF) and kept in ACSF at room temperature for at least one hr before recording, as described (Zhu et al., 2005). Slices were maintained in an interface-type chamber perfused with oxygenated ACSF (95% O2 and 5% CO₂) containing in mM: 124 NaCl, 2.0 KCl, 1.3 MgSO₄, 2.5 CaCl₂, 1.2 KH₂PO₄, 25 NaHCO₃, and 10 glucose (2-3 ml/min). Bipolar stimulating electrodes were placed in the CA1 stratum radiatum to stimulate Schaffer collateral and commissural fibers. Field potentials were recorded using ACSF-filled micropipettes at 28-29° C. The recording electrodes were placed in the stratum radiatum for field excitatory postsynaptic potentials (fEPSPs), and stratum pyramidale for population spikes. The stimulus strength of the 0.1 ms pulses was adjusted to evoke 30-35% of maximum response for fEPSPs, and 50% of maximal response for population spikes. A stable baseline of responses was established for at least 30 min at 0.033 Hz. Tetanic LTP was induced by high-frequency stimulation in brief trains (100 Hz, 1 s), applied either as a single train or four trains separated by 5 min intervals. A short train consisted of 5 stimuli (100 Hz within-burst). When indicated, ACSF was supplemented with anisomycin (Calbiochem, Calif.), PKRi (Calbiochem, Calif.), bicuculline (Tocris) or diazepam (Sigma-Aldrich). It should be noted that the inventors used bicuculline free base which only blocks GABAA receptor rather than bicuculline-M (bicuculline methiodide, methobromide or methochloride) which in addition to GABAA receptor also blocks small conductance (SK) calcium-activated potassium channels (Debarbieux et al., 1998). PKRi was used at a final concentration of 1 μM (0.01% DMSO), which is known to block PKR activity ex-vivo (Page et al., 2006; Wang et al., 2007). To reduce day-to-day variations, whenever possible simultaneous recordings (in the same chamber) were obtained from slices from Pkr^{-/-} mice and WT littermates treated with drugs or vehicle. Statistical analysis was performed using t-test and two-way ANOVA. All data are presented as means±SEM and “n” indicates the number of slices.

Whole Cell Recording:

Horizontal hippocampal slices were cut as described above. All recordings were at 28-29° C. using conventional patch-clamp techniques and an Axopatch 200B amplifier (Molecular Devices, Union City, Calif.). CA neurons were visually identified by infrared differential interference contrast video microscopy on the stage of an upright microscope (Axioskope FS2, Carl Zeiss, Oberkochen, Germany). Patch pipettes (resistances 4-6 MΩ) were filled with (in mM): 110 K-gluconate, 10 KCI, 10 HEPES, 10 Na₂-phosphocreatine, 2 Mg₃-ATP, 0.2 Na₃-GTP; pH was adjusted to 7.2 and osmolarity to 290 mOsm using a Wescor 5500 vapor pressure osmometer (Wescor, Logan, Utah). Synaptic responses were evoked with a bipolar stimulating electrode positioned in striatum radiatum. Gluconate was replaced with KCl for spontaneous inhibitory postsynaptic currents (sIPSCs). sIPSCs were recorded in the presence of 2 mM kynurenic acid while miniature IPSCs were recorded in the presence of kynurenic acid (2 mM) and tetrodotoxin (TTX; 1 M). Evoked IPSCs were recorded in the presence or absence of D-AP5 (50 μM), CNQX (10 μM) and CGP55845 (10 μM). Excitatory postsynaptic currents (EPSCs) were recorded in the presence of 10 μM bicuculline or 100 μM picrotoxin. The electrical signals were filtered on-line at 5 kHz and digitized at 10 kHz. Series resistance (Rs) and input resistance (Ri) were measured continually during recording with the application of a −5 mV×25 ms test pulse prior to stimulation. If Rs ever varied more than ±20%, the recording was abandoned and the data were discarded. All drugs were obtained from Tocris (Ellisville, Mo.). PKRi was used at a final concentration of 1 μM.

### Contextual and Auditory Fear Conditioning

The experimenter was blind to the genotype for all behavioral tests. Fear conditioning was performed as previously described (Costa-Mattioli et al., 2007). Mice were first handled for 3-5 min for 3 days and then habituated to the conditioning chamber for 20 min for another 3 days. On the training day, after 2 min in the conditioning chamber, mice received a pairing of a tone (2800 Hz, 85 db, 30 s) with a co-terminating foot-shock (0.35 mA, 1 s), after which they remained in the chamber for two additional min and then were returned to their home cages. Mice were tested 24 hr after training for “freezing” (immobility with the exception of respiration) in response to the tone (in a chamber to which they had not been conditioned) and to the training context (training chamber).

During testing for auditory fear conditioning, mice were placed in the chamber and freezing responses were recorded during the initial 2 min (pre-CS period) and during the last 3 min when the tone was played. Mice were returned to their cages 30 s after the end of the tone. For testing contextual fear conditioning, mice were returned to the conditioning chamber for 5 min. For extinction trials, freezing in response to the conditioned context was assessed for 5 min, 24 hr, 48 hr, 72 hr and 96 hr after training and normalized to the amount of freezing obtained at 24 hr. For all tests, freezing behavior was determined at 5 s intervals during a 5 min period. The percent of time spent by the mouse freezing was taken as an index of learning and memory. PKRi was freshly dissolved in saline and then i.p-injected immediately after fear conditioning, at a dose of 0.1 mg/kg, which is known to block PKR activity in the hippocampus in vivo (Ingrand et al., 2007). Statistical analysis was based on repeated measures ANOVA and between-group comparisons by Tukey's Test.

### Morris Water Maze

Tests were performed in a circular pool of opaque water, as previously described (Moris et al., 1982). WT and I littermates were trained using a relatively weak training protocol, one trial per day (Costa-Mattioli et al., 2007). The latencies of escape from the water onto the hidden (submerged) platform were monitored by an automated video tracking system (HVS Image, Buckingham, UK). For the probe trial, the platform was removed from the pool and the animals were allowed to search for 60 s. The % of time spent in each quadrant of the pool (quadrant occupancy) was recorded. There was no significant difference in swimming speed between WT and Pkr^{-/-} mice. The animals were trained at the same time of day during their animals' light phase. The statistical analysis was based on repeated measures ANOVA and between-group comparisons by Tukey's Test.

### Elevated Plus Maze Test

The elevated plus-maze apparatus consisted of two open arms (35×5 cm) and two enclosed arms of the same size (with 15 cm high opaque walls). The arms and central square were made of plastic plates and were elevated 40 cm above the floor. Mice were placed in the central square of the maze (5×5 cm). Behavior was recorded during a 5-min period. Data acquisition and analysis were performed automatically with ANYMAZE software.

### Immunohistochemistry and Western Blotting

Hippocampal cell lysates, Western blotting and immunohistochemistry were performed as previously described (Costa-Mattioli et al., 2007). Mice were deeply anesthetized and perfused intracardially with cold PBS and subsequently with 4% paraformaldehyde (PFA) in ice cold 0.1 M phosphatase buffer (PBS). Brains were removed from the skull, stored in a 4% PFA solution overnight (at 4° C.), and 40 μm horizontal sections were cut on a microtome (Leica VT1000S, Germany). Free-floating method was used while rinsing between steps. Sections were first placed in a blocking solution (5% BSA, 0.3% Triton and 4% Normal Goat Serum in phosphate buffered saline) at room temperature for one hour, incubated overnight with primary antibodies [PKR (Santa Cruz Biotechnology, CA), GAD67 (Millipore, Billerica, Mass.), V-Glut 1 (Synaptic Systems, Goettingen, Germany) and PSD95 (NeuroMab, CA)] and then rinsed four times (for 20 min) with PBS before incubation with the secondary antibody (for 4 hr). After four washes (each for 20 min) with PBS, the sections were mounted on Superfrost® Plus slides (VWR, West Chester, Pa.). Finally, the sections were cover-slipped with VECTASHIELD Hard Set mounting medium (Vector Lab, Burlingame, Calif.). Digital photos were taken with a Zeiss LSM 510 laser confocal microscope.

Egr-1 Staining:

Prior to contextual fear conditioning, WT and Pkr^{-/-} mice (n=6 both groups) were handled for three consecutive days. They were then trained as described above. Control groups were exposed to the context, except that they received no shock during training. Ninety minutes following training, brain sections were cut as described above and pre-treated in 0.3% H₂O₂ in PBS. The sections were then incubated with an anti Egr-1 (1:7500) primary rabbit polyclonal antibody (Cell Signaling Technologies, Denver, Mass.) in a blocking solution (1% BSA, 0.3% triton and 4% normal goat serum in PBS) for 48 hr; and then incubated for 60 min at room temperature with a biotinylated goat-anti rabbit antibody (1:500; Vector Laboratories, Burlingame, Calif.) followed by an avidin-biotin-horseradish peroxidase (HRP; ABC kit; Vector Laboratories, Burlingame, Calif.). The bound peroxidase was located by incubating sections in 0.1% 3,3′-diaminobenzidine (DAB) and 0.025% H₂O₂ at room temperature for 5-10 min, which generated the visible substrate. Immunoreactive CA1 neurons were counted within a given area (0.07 mm²), as described earlier (Frankland et al., 2004; Hall et al., 2001).

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PUBLICATIONS

Abraham, N. et al. Characterization of transgenic mice with targeted disruption of the catalytic domain of the double-stranded RNA-dependent protein kinase, PKR. J Biol Chem 274, 5953-62 (1999).
Abraham, W. C., Gustafsson, B. & Wigstrom, H. Single high strength afferent volleys can produce long-term potentiation in the hippocampus in vitro. Neurosci Lett 70, 217-22 (1986).
Bando, Y. et al. Double-strand RNA dependent protein kinase (PKR) is involved in the extrastriatal degeneration in Parkinson's disease and Huntington's disease. Neurochem Int 46, 11-8 (2005).
Baron, R. et al. IFN-gamma enhances neurogenesis in wild-type mice and in a mouse model of Alzheimer's disease. FASEB J 22, 2843-52 (2008).
Beenhakker, M. P. & Huguenard, J. R. Neurons that fire together also conspire together: is normal sleep circuitry hijacked to generate epilepsy? Neuron 62, 612-32 (2009).
Ben-Asouli, Y., Banai, Y., Pel-Or, Y., Shir, A. & Kaempfer, R. Human interferon-gamma mRNA autoregulates its translation through a pseudoknot that activates the interferon-inducible protein kinase PKR. Cell 108, 221-32 (2002).
Bryant, E. Genius and Epilepsy. Brief sketches of Great Men Who Had Both (Ye Old Depot Press, Concord, Mass., 1953).
Buzsaki, G. Rythms of the Brain, (Oxford University Press, New York, 2006).
Carnevalli, L. S. et al. Phosphorylation of the alpha subunit of translation initiation factor-2 by PKR mediates protein synthesis inhibition in the mouse brain during status epilepticus. Biochem J 397, 187-94 (2006).
Chang, R. C., Wong, A. K., Ng, H. K. & Hugon, J. Phosphorylation of eukaryotic initiation factor-2 alpha (elF2alpha) is associated with neuronal degeneration in Alzheimer's disease. Neuroreport 13, 2429-32 (2002).
Cohen-Chalamish, S. et al. Dynamic refolding of IFN-gamma mRNA enables it to function as PKR activator and translation template. Nat Chem Biol 5, 896-903 (2009).
Costa-Mattioli, M. et al. elF2alpha phosphorylation bidirectionally regulates the switch from short- to long-term synaptic plasticity and memory. Cell 129, 195-206 (2007).
Costa-Mattioli, M., Sossin, W. S., Klann, E. & Sonenberg, N. Translational Control of Long-Lasting Synaptic Plasticity and Memory. Neuron 61, 10-26 (2009).
Couturier, J. et al. Interaction of double-stranded RNA-dependent protein kinase (PKR) with the death receptor signaling pathway in amyloid beta (Abeta)-treated cells and in APPSLPS1 knock-in mice. J Biol Chem 285, 1272-82 (2010).
Davies, C. H., Starkey, S. J., Pozza, M. F. & Collingridge, G. L. GABA autoreceptors regulate the induction of LTP. Nature 349, 609-11 (1991).
Debarbieux, F., Brunton, J. & Charpak, S. Effect of bicuculline on thalamic activity: a direct blockade of IAHP in reticularis neurons. J Neurophysiol 79, 2911-8 (1998).
Dever, T. E., Dar, A. C. & Sicheri, F. The elF2alpha kinases. in Translational Control in Biology and Medicine (eds. Mathews, M. B., Sonenberg, N. & Hershey, J. W. B.) 319-45 (Cold Spring Harbor Laboratory Press, Cold Spring Harbor, N.Y., 2007).
Frankland, P. W., Bontempi, B., Talton, L. E., Kaczmarek, L. & Silva, A. J. The involvement of the anterior cingulate cortex in remote contextual fear memory. Science 304, 881-3 (2004).
Freund, T. F. Interneuron Diversity series: Rhythm and mood in perisomatic inhibition. Trends Neurosci 26, 489-95 (2003).
Garcia, M. A., Meurs, E. F. & Esteban, M. The dsRNA protein kinase PKR: virus and cell control. Biochimie 89, 799-811 (2007).
Getts, D. R. et al. Role of IFN-gamma in an experimental murine model of West Nile virus-induced seizures. J Neurochem 103, 1019-30 (2007).
Girardeau, G., Benchenane, K., Wiener, S. I., Buzsaki, G. & Zugaro, M. B. Selective suppression of hippocampal ripples impairs spatial memory. Nat Neurosci 12, 1222-3 (2009).
Grossberg, S. E. & Kawade, Y. The expression of potency of neutralizing antibodies for interferons and other cytokines. Biotherapy 10, 93-8 (1997).
Gusev, P. A. & Alkon, D. L. Intracellular correlates of spatial memory acquisition in hippocampal slices: long-term disinhibition of CA1 pyramidal cells. J Neurophysiol 86, 881-99 (2001).
Haefely, W. The GABA-benzodiazepine interaction fifteen years later. Neurochem Res 15, 169-74 (1990).
Hall, J., Thomas, K. L. & Everitt, B. J. Rapid and selective induction of BDNF expression in the hippocampus during contextual learning. Nat Neurosci 3, 533-5 (2000).
Hall, J., Thomas, K. L. & Everitt, B. J. Fear memory retrieval induces CREB phosphorylation and Fos expression within the amygdala. Eur J Neurosci 13, 1453-8 (2001).
Han, J. H. et al. Neuronal competition and selection during memory formation. Science 316, 457-60 (2007).
Han, J. H. et al. Selective erasure of a fear memory. Science 323, 1492-6 (2009).
Hangya, B., Borhegyi, Z., Szilagyi, N., Freund, T. F. & Varga, V. GABAergic neurons of the medial septum lead the hippocampal network during theta activity. J Neurosci 29, 8094-102 (2009).
Heaton, P. & Wallace, G. L. Annotation: the savant syndrome. J Child Psychol Psychiatry 45, 899-911 (2004).
Holmes, G. L. & Lenck-Santini, P. P. Role of interictal epileptiform abnormalities in cognitive impairment. Epilepsy Behav 8, 504-15 (2006).
Hughes, J. R. A review of Savant Syndrome and its possible relationship to epilepsy. Epilepsy Behav 17, 147-52 (2010).
Huguenard, J. R. & McCormick, D. A. Thalamic synchrony and dynamic regulation of global forebrain oscillations. Trends Neurosci 30, 350-6 (2007).
Ingrand, S. et al. The oxindole/imidazole derivative C16 reduces in vivo brain PKR activation. FEBS Lett 581, 4473-8 (2007).
Izquierdo, I. & Medina, J. H. GABAA receptor modulation of memory: the role of endogenous benzodiazepines. Trends Pharmacol Sci 12, 260-5 (1991).
Jammi, N. V., Whitby, L. R. & Beal, P. A. Small molecule inhibitors of the RNA-dependent protein kinase. Biochem Biophys Res Commun 308, 50-7 (2003).
Jones, M. W. et al. A requirement for the immediate early gene Zif268 in the expression of late LTP and long-term memories. Nat Neurosci 4, 289-96 (2001).
Kandel, E. R. The molecular biology of memory storage: a dialogue between genes and synapses. Science 294, 1030-8 (2001).
Klausberger, T. & Somogyi, P. Neuronal diversity and temporal dynamics: the unity of hippocampal circuit operations. Science 321, 53-7 (2008).
LeDoux, J. E. Emotion circuits in the brain. Annu Rev Neurosci 23, 155-84 (2000).
Lee, Y. S. & Silva, A. J. The molecular and cellular biology of enhanced cognition. Nat Rev Neurosci 10, 126-40 (2009).

Mann, E. O. & Mody, I. Control of hippocampal gamma oscillation frequency by tonic inhibition and excitation of interneurons. Nat Neurosci 13, 205-12 (2010).
Mann, E. O. & Paulsen, O. Role of GABAergic inhibition in hippocampal network oscillations. Trends Neurosci 30, 343-9 (2007).
Maurer, A. P. & McNaughton, B. L. Network and intrinsic cellular mechanisms underlying theta phase precession of hippocampal neurons. Trends Neurosci 30, 325-33 (2007).
McCormick, D. A. & Contreras, D. On the cellular and network bases of epileptic seizures. Annu Rev Physiol 63, 815-46 (2001).
McGaugh, J. L. & Roozendaal, B. Drug enhancement of memory consolidation: historical perspective and neurobiological implications. Psychopharmacology (Berl) 202, 3-14 (2009).
Morel, M., Couturier, J., Lafay-Chebassier, C., Paccalin, M. & Page, G. PKR, the double stranded RNA-dependent protein kinase as a critical target in Alzheimer's disease. J Cell Mol Med 13, 1476-88 (2009).
Morris, R. G., Garrud, P., Rawlins, J. N. & O'Keefe, J. Place navigation impaired in rats with hippocampal lesions. Nature 297, 681-3 (1982).
Muller, M., Fontana, A., Zbinden, G. & Gahwiler, B. H. Effects of interferons and hydrogen peroxide on CA3 pyramidal cells in rat hippocampal slice cultures. Brain Res 619, 157-62 (1993).
Niedermeyer, E. Epileptic seizure Disorders. in Electroengcephalography: Bacis principles, Clinical Application, And Related Fields, Vol. Fifth edition (ed. Niedermeyer, E.F.L.D.S.) 505-621 (Lippincott Williams and Wilkins, Philadelphia, 2005).
Noebels, J. L. The biology of epilepsy genes. Annu Rev Neurosci 26, 599-625 (2003).
Page, G. et al. Activated double-stranded RNA-dependent protein kinase and neuronal death in models of Alzheimer's disease. Neuroscience 139, 1343-54 (2006).
Palop, J. J. et al. Aberrant excitatory neuronal activity and compensatory remodeling of inhibitory hippocampal circuits in mouse models of Alzheimer's disease. Neuron 55, 697-711 (2007).
Paquet, C. et al. Neuronal phosphorylated RNA-dependent protein kinase in Creutzfeldt-Jakob disease. J Neuropathol Exp Neurol 68, 190-8 (2009).
Paulsen, O. & Moser, E. I. A model of hippocampal memory encoding and retrieval: GABAergic control of synaptic plasticity. Trends Neurosci 21, 273-8 (1998).
Peel, A. L. & Bredesen, D. E. Activation of the cell stress kinase PKR in Alzheimer's disease and human amyloid precursor protein transgenic mice. Neurobiol Dis 14, 52-62 (2003).
Peel, A. L. et al. Double-stranded RNA-dependent protein kinase, PKR, binds preferentially to Huntington's disease (HD) transcripts and is activated in HD tissue. Hum Mol Genet. 10, 1531-8 (2001).
Price, M. G. et al. A triplet repeat expansion genetic mouse model of infantile spasms syndrome, Arx(GCG)10+7, with interneuronopathy, spasms in infancy, persistent seizures, and adult cognitive and behavioral impairment. J Neurosci 29, 8752-63 (2009).
Remington's Pharmaceutical Sciences, 18th Ed. Mack Printing Company, 1990.
Renno, T. et al. Interferon-gamma in progression to chronic demyelination and neurological deficit following acute EAE. Mol Cell Neurosci 12, 376-89 (1998).
Scheuner, D. et al. Translational control is required for the unfolded protein response and in vivo glucose homeostasis. Mol Cell 7, 1165-76 (2001).
Shimazawa, M. & Hara, H. Inhibitor of double stranded RNA-dependent protein kinase protects against cell damage induced by ER stress. Neurosci Lett 409, 192-5 (2006).
Shirvalkar, P. R., Rapp, P. R. & Shapiro, M. L. Bidirectional changes to hippocampal theta-gamma comodulation predict memory for recent spatial episodes. Proc Natl Acad Sci USA 107, 7054-9.
Silva, A. J., Zhou, Y., Rogerson, T., Shobe, J. & Balaji, J. Molecular and cellular approaches to memory allocation in neural circuits. Science 326, 391-5 (2009).
Sohal, V. S., Zhang, F., Yizhar, O. & Deisseroth, K. Parvalbumin neurons and gamma rhythms enhance cortical circuit performance. Nature 459, 698-702 (2009).
Steriade, M. Cellular substrates of brain rhythms. in Electroengcephalography: Bacis principles, Clinical Application, And Related Fields, Vol. Fifth edition (ed. Niedermeyer, E.F.L.D.S.) 505-621 (Lippincott Williams and Wilkins, Philadelphia, 2005).
Steriade, M. Neuronal substrates of Sleep and Epilepsy, (Cambridge University Press, Cambridge, 2003).
Thomson, A. M. Facilitation, augmentation and potentiation at central synapses. Trends Neurosci 23, 305-12 (2000).
Vingerhoets, G. Cognitive effects of seizures. Seizure 15, 221-6 (2006).
Wang, X., Fan, Z., Wang, B., Luo, J. & Ke, Z. J. Activation of double-stranded RNA-activated protein kinase by mild impairment of oxidative metabolism in neurons. J Neurochem 103, 2380-90 (2007).
Wigstrom, H. & Gustafsson, B. Facilitated induction of hippocampal long-lasting potentiation during blockade of inhibition. Nature 301, 603-4 (1983).

Zhou, Y. et al. CREB regulates excitability and the allocation of memory to subsets of neurons in the amygdala. Nat Neurosci 12, 1438-43 (2009).
Zhu, P. J., Stewart, R. R., McIntosh, J. M. & Weight, F. F. Activation of nicotinic acetylcholine receptors increases the frequency of spontaneous GABAergic IPSCs in rat basolateral amygdala neurons. J Neurophysiol 94, 3081-91 (2005).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of enhancing cognition in an individual, comprising the step of providing to the individual a therapeutically effective amount of an inhibitor of double-stranded RNA-protein dependent kinase.

2. The method of claim 1, wherein the inhibitor comprises a protein, nucleic acid, or small molecule.

3. The method of claim 1, wherein the inhibitor comprises a small molecule.

4. The method of claim 1, wherein the individual has no detectable cognitive dysfunction.

5. The method of claim 1, wherein the individual has Alzheimer's Disease, Parkinson's Disease, or is elderly.

6. The method of claim 1, wherein the inhibitor has the general formula: 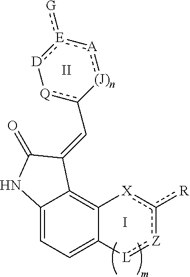
wherein
X is H, OH, SH, O, S, N, NH, CH, CH₂, or C═O
Z is H, OH, SH, O, S, N, NH, CH, CH₂, or C═O,
R is H; O, NH₂; or OH
Y is CH₂; CH, N, NH, C, or O
L is H, OH, SH, O, S, N, NH, CH, CH₂, or C═O
m is 0 or 1
wherein when L is H, OH or SH and X is H, OH or SH, then Z, Y and R are not present;
wherein when X is H, OH or SH and Z is H, OH or SH, then Y and R are not present;
wherein when X is N or CH, then X forms a double bond with Y;
wherein when X is O, S, NH, CH₂ or C═O, then X forms a single bond with Y;
wherein when X forms a double bond with Y; Z forms a single bond with Y;
wherein when Z forms a double bond with Y; X forms a single bond with Y;
wherein when Z is N or CH, then Z forms a double bond with Y;
wherein when Z is O, S, NH, CH₂ or C═O, then Z forms a single bond with Y
wherein when Y is C; R is H, OH or NH₂; or R is O and forms a double bond with Y
A is H, OH, SH, O, S, N, NH, CH, CH₂, or C═O
D is H, OH, SH, O, S, N, NH, CH, CH₂, or C═O
E is CH₂, CH, N, NH, C, or O
G is H; O, NH₂; or OH;
J is H, OH, SH, O, S, N, NH, CH, CH₂, or C═O
Q is CH₂, CH, N, NH, or O
n is 0 or 1
wherein when J is H, OH or SH and D is H, OH or SH, then A, E and G are not present;
wherein when D is H, OH or SH and A is H, OH or SH, then E and G are not present;
wherein when D is N or CH, then D forms a double bond with E;
wherein when D is O, S, NH, CH₂ or C═O, then D forms a single bond with E;
wherein when D forms a double bond with E; A forms a single bond with E;
wherein when A forms a double bond with E; D forms a single bond with E;
wherein when A is N or CH, then A forms a double bond with E;
wherein when A is O, S, NH, CH₂ or C═O, then A forms a single bond with E
wherein when E is C; G is H, OH or NH₂; or G is O and forms a double bond with E; and,
wherein the composition is a pharmaceutically acceptable salt or hydrate thereof.

7. The method of claim 6, wherein m is 0; X is NH; Y is C; Z is N; and, R is SH.

8. The method of claim 6, wherein m is 0; X is S; Y is CH₂; Z is S.

9. The method of claim 6, wherein m is 0; X is NH; Y is O; and Z is CH₂.

10. The method of claim 6, wherein m is 0; X is NH; Y is C; R is 0; and Z is NH.

11. The method of claim 6, wherein m is 0; X is C═O; Y is NH and Z is C═O.

12. The method of claim 6, wherein m is 0; X is S; Y is N and Z is CH.

13. The method of claim 6, wherein m is 1; X is N; Y is CH; Z is CH and L is N.

14. The method of claim 6, wherein m is 1; X is S; Y is CH₂, Z is CH₂, and L is NH.

15. The method of claim 6, wherein n is 0; Q is CH, D is S; E is CH and A is N.

16. The method of claim 6, wherein n is 0, Q is CH; D is N; E is CH; and A is S.

17. The method of claim 6, wherein n is 0, Q is N; D is O; E is CH; A is CH.

18. The method of claim 6, wherein n is 0; Q is CH, D is NH; E is C, G is O and A is NH.

19. The method of claim 6, wherein n is 0, Q is CH; D is CH; E is CH; and A is NH.

20. The method of claim 6, wherein n is 0; Q is CH; D is NH; E is C; and A is C.

21. The method of claim 6, wherein n is 1; Q is CH₂; D is O; E is CH₂; A is CH₂; and, J is NH.

22. The method of claim 6, wherein n is 1; Q is CH; D is CH; E is CH; A is CH; and, J is N.

23. The method of claim 6, wherein the composition is selected from the group consisting of: 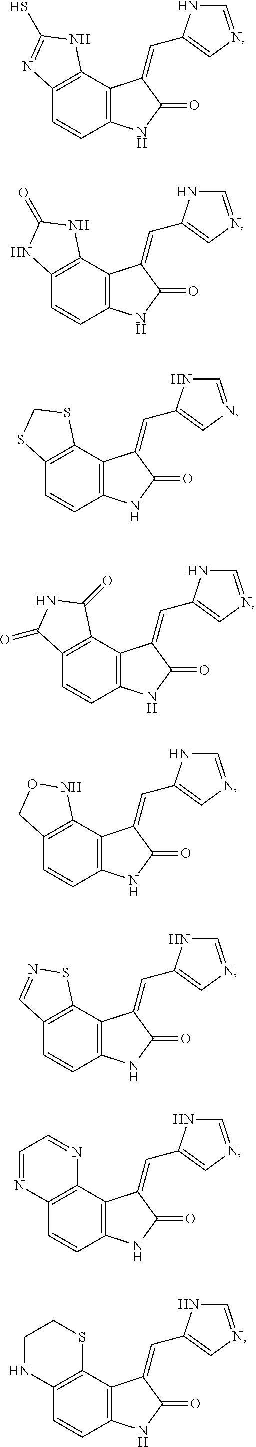 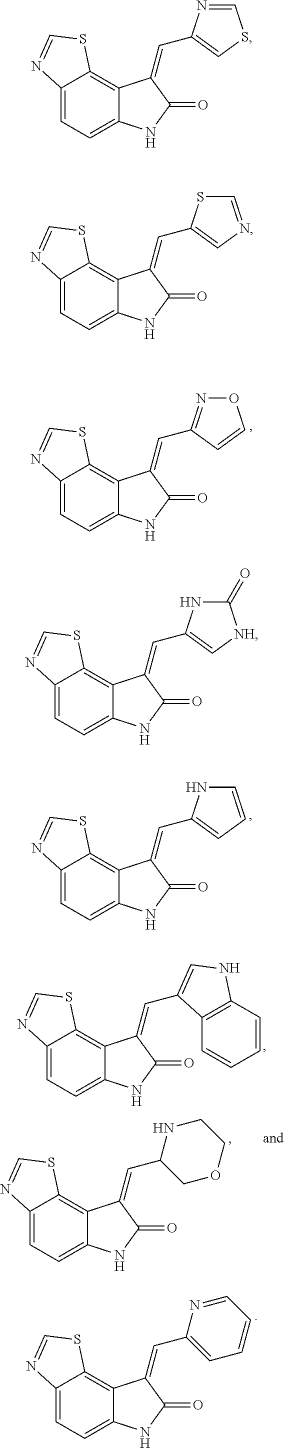
and or a combination thereof.

24. The method of claim 1, wherein the enhancement of cognition is further defined as enhancing memory in the individual.
